(19) United States
(12) Patent Application Publication
(10) Pub. No.: US 2021/0123919 A1
(43) Pub. Date: Apr. 29, 2021

(54) BIOMARKERS FOR A COMBINATION THERAPY COMPRISING LENVATINIB AND A PD-1 ANTAGONIST

(71) Applicants: Merck Sharp & Dohme Corp., Rahway, NJ (US); Eisai R&D Management Co., Ltd., Bunkyo-ku, Tokyo (JP)

(72) Inventors: Yasuhiro FUNAHASHI, Bunkyo-ku, Tokyo (JP); Kotaro KODAMA, Tsuchiura-shi, Ibaraki (JP); Ryo DAIRIKI, Tsukuba-shi, Ibaraki (JP); Yukinori MINOSHIMA, Tsukuba-shi, Ibaraki (JP)

(73) Assignees: Merck Sharp & Dohme Corp., Rahway, NJ (US); Eisai R&D Management Co., Ltd., Bunkyo-ku, Tokyo (JP)

(21) Appl. No.: 17/052,133

(22) PCT Filed: May 13, 2019
(86) PCT No.: PCT/US2019/031967

§ 371 Date: Oct. 30, 2020

Related U.S. Application Data
(60) Provisional application No. 62/670,984, filed on May 14, 2018.

(51) Int. Cl. G01N 33/574; A61K 31/47; A61K 39/395; C07K 16/28; A61P 35/00

(52) U.S. Cl. CPC G01N 33/57442; A61K 31/47; C07K 2317/24; C07K 16/2818; A61P 35/00; A61K 39/3955

(57) ABSTRACT

Biomarkers are provided that are predictive of a subject's responsiveness to a combination therapy comprising lenvatinib compound and a PD-1 antagonist. The biomarkers, compositions, and methods described herein are useful in selecting appropriate treatment modalities for a subject having, suspected of having, or at risk of developing cancer.

### FIELD

Disclosed herein are biomarkers for identifying populations of cancer subjects who respond to a combination therapy comprising lenvatinib and a Programmed Cell Death 1 protein (PD-1) antagonist.

### BACKGROUND

A number of kinase inhibitors have been developed as antitumor agents. For example, a group of compounds having inhibitory activity against receptor tyrosine kinases, such as vascular endothelial growth factor receptor (VEGFR), are known to inhibit angiogenesis and are regarded as a new class of antitumor agents. Lenvatinib mesylate (also known as E7080) is an oral tyrosine kinase inhibitor targeting VEGFR1-3, fibroblast growth factor receptor (FGFR) 1-4, rearranged during transfection receptor (RET), KIT, and platelet-derived growth factor receptor (PDGFR). Lenvatinib mesylate has been approved as LENVIMA® by U.S. Food and Drug Administration (USFDA) for the treatment of patients with locally recurrent or metastatic, progressive, radioactive iodine-refractory differentiated thyroid cancer and in combination with everolimus, for patients with advanced renal cell cancer following one prior anti-angiogenic therapy.

PD-1 is recognized as an important player in immune regulation and the maintenance of peripheral tolerance. PD-1 is moderately expressed on naive T-, B- and Natural killer T (NKT)-cells and up-regulated by T/B-cell receptor signaling on lymphocytes, monocytes and myeloid cells (1).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, PD-L1 expression has been demonstrated to correlate with poor prognosis and reduced overall survival irrespective of subsequent treatment in some tumors (2-13). Similarly, PD-1 expression on tumor infiltrating lymphocytes was found to mark dysfunctional T-cells in breast cancer and melanoma (14-15) and to correlate with poor prognosis in renal cancer (16). It has been proposed that PD-L1 expressing tumor cells interact with PD-1 expressing T cells to attenuate T cell activation and evasion of immune surveillance, thereby contributing to an impaired immune response against the tumor. Therefore, an antibody directed against either the PD-1 receptor or the PD-L1 ligand can inhibit the binding there between, resulting in an increased immune action on the tumor cells (23).

Several monoclonal antibodies that inhibit the interaction between PD-1 and one or both of its ligands, PD-L1 and PD-L2, are approved by the United States Food and Drug Administration (USFDA) and/or in clinical development for treating cancer. It has been proposed that the efficacy of such antibodies might be enhanced if administered in combination with other approved or experimental cancer therapies, e.g., radiation, surgery, chemotherapeutic agents, targeted therapies, agents that inhibit other signaling pathways that are dysregulated in tumors, and other immune enhancing agents.

Most anti-tumor treatments are associated with undesirable side effects, such as profound nausea, vomiting, or severe fatigue. Also, while anti-tumor treatments have been successful, many treatments do not produce significant clinical responses in all patients who receive them resulting in undesirable side effects, delays, and costs associated with ineffective treatment. For patients who are unresponsive to therapies, significant expenditures in their treatment may be made before determination of responsiveness can be made. Therefore, new methods are needed to identify responsive patients from unresponsive patients early in therapy. Biomarkers that can be used to predict the response of a subject to an antitumor agent prior to or during the administration of are greatly needed. In addition, it is useful to have biomarkers that can be used to evaluate whether therapy comprising an antitumor agent is effective.

### SUMMARY

The present application is based, at least in part, on the identification of biomarkers that are predictive of a cancer subject's responsiveness to a combination therapy comprising lenvatinib or a pharmaceutically acceptable salt thereof (hereinafter referred to as “a lenvatinib compound”) and a PD-1 antagonist. The ratio of levels of one or more proteins selected from the group consisting of interferon (IFN)-γ, IL-10, CXCL9, CXCL10, CXCL11, CXCL12, FGF-19, and FGF-23 of pre- and post-treatment with a combination therapy comprising a lenvatinib compound and a PD-1 antagonist can be useful in determining the likelihood that a subject having cancer selected from the group consisting of: endometrial cancer, non-small cell lung cancer, renal cell carcinoma, urothelial cancer, head and neck cancer, melanoma, bladder cancer, hepatocellular carcinoma, breast cancer, ovarian cancer, gastric cancer, colorectal cancer, glioblastoma, biliary tract cancer and thyroid cancer that will respond to continued combination therapy with the lenvatinib compound and a PD-1 antagonist.

The application also provides methods for evaluating whether to continue treatment with a lenvatinib compound and a PD-1 antagonist for a subject having cancer selected from the group consisting of: an endometrial cancer, a non-small cell lung cancer (NSCLC), a renal cell carcinoma ((RCC), e.g. clear cell RCC, non-clear cell RCC), a urothelial cancer, a head and neck cancer (e.g. head and neck squamous cell cancer), a melanoma (e.g., advanced melanoma such as Stage III-IV high-risk melanoma, unresectable or metastatic melanoma), a bladder cancer, a hepatocellular carcinoma, a breast cancer (e.g., triple negative breast cancer, ER⁺/HER2⁻breast cancer), an ovarian cancer, a gastric cancer (e.g. metastatic gastric cancer or gastroesophageal junction adenocarcinoma), a colorectal cancer, a glioblastoma, a biliary tract cancer, a glioma (e.g., recurrent malignant glioma with a hypermutator phenotype), Merkel cell carcinoma (e.g., advanced or metastatic Merkel cell cancer),

Hodgkin lymphoma, non-Hodgkin lymphoma (e.g. primary mediastinal B-cell lymphoma (PMBCL)), a cervical cancer, an advanced or refractory solid tumor, a small cell lung cancer (e.g., stage IV non-small cell lung cancer), a non-squamous non-small cell lung cancer, desmoplastic melanoma, a pediatric advanced solid tumor or lymphoma, a mesothelin-positive pleural mesothelioma, an esophageal cancer, an anal cancer, a salivary cancer, a prostate cancer, a carcinoid tumor, a primitive neuroectodermal tumor (pNET), and a thyroid cancer. An exemplary group of cancers for such method also contemplates a cancer is selected from: a melanoma, non-small cell lung cancer (NSCLC), a head and neck cancer, Hodgkin lymphoma, PMBCL, a urothelial carcinoma, a gastric cancer, a cervical cancer, a hepatocellular carcinoma, a

Merkel cell carcinoma, a thyroid cancer, and an endometrial cancer. Low or high levels of one or more proteins selected from the group consisting of IFN-γ, IL-10, CXCL9, CXCL10, CXCL11, CXCL12, FGF-19, and FGF-23 before and/or after treatment with the combination therapy can be useful in evaluating whether to continue treatment with lenvatinib compound and a PD-1 antagonist. For example, higher ratios of levels of one or more proteins selected from the group consisting of IFN-y, IL-10, CXCL9, CXCL10, CXCL11 CXCL12, FGF-19, and FGF-23 (post/pre-treatment with the combination therapy) compared to control ratios from samples of patients who are known to not respond to such combination therapy (negative control) can be useful in assessing/evaluating whether the test subject will benefit from continued the combination therapy. Also, higher ratios of levels of one or more proteins selected from the group consisting of IFN-y, IL-10, CXCL9, CXCL10, CXCL11 CXCL12, FGF-19, and FGF-23 (post/pre-treatment with the combination therapy) compared to control ratios from samples of patients who are known to respond to such combination therapy (positive control) can be useful in assessing/evaluating whether the test subject will benefit from continued the combination therapy.

Thus, the biomarkers and compositions described herein are useful, for example, in identifying and/or selecting a patient or a subset of patients having cancer selected from the group consisting of endometrial cancer, non-small cell lung cancer, renal cell carcinoma, urothelial cancer, head and neck cancer, melanoma, bladder cancer, hepatocellular carcinoma, breast cancer, ovarian cancer, gastric cancer, colorectal cancer, glioblastoma, biliary tract cancer, and thyroid cancer that could benefit from treatment with lenvatinib compound and a PD-1 antagonist. In addition, the methods described herein are useful, for example, in selecting appropriate treatment modalities (e.g., a combination therapy comprising a lenvatinib compound and a PD-1 antagonist) for a subject suffering from, suspected of having, or at risk of developing cancer selected from the group consisting of endometrial cancer, non-small cell lung cancer, renal cell carcinoma, urothelial cancer, head and neck cancer, melanoma, bladder cancer, hepatocellular carcinoma, breast cancer, ovarian cancer, gastric cancer, colorectal cancer, glioblastoma, biliary tract cancer, and thyroid cancer. Also, the methods allow a health care practitioner to determine whether to continue with a combination therapy comprising a lenvatinib compound and a PD-1 antagonist or change therapies and use a different treatment.

In one aspect, the application provides a method of predicting the response of a subject having, suspected of having, or at risk of developing, at least one cancer selected from the group consisting of: endometrial cancer, non-small cell lung cancer, renal cell carcinoma, urothelial cancer, head and neck cancer, melanoma, bladder cancer, hepatocellular carcinoma, breast cancer, ovarian cancer, gastric cancer, colorectal cancer, glioblastoma, biliary tract cancer, and thyroid cancer to a combination therapy comprising a lenvatinib compound and a PD-1 antagonist. This method can also be used to evaluate/assess the benefit of continued administration of a combination therapy comprising a lenvatinib compound and a PD-1 antagonist. The method involves providing a biological sample from the subject, e.g., a blood sample obtained from the subject before the therapy comprising a lenvatinib compound and a PD-1 antagonist (pre-treatment); providing a blood sample obtained from the subject after initiation of the therapy comprising a lenvatinib compound and a PD-1 antagonist (post-treatment);

measuring the levels of one or more proteins selected from the group consisting of IFN-γ, IL-10, CXCL9, CXCL10, CXCL11, CXCL12, FGF-19, and FGF-23 in the pre-treatment biological sample and the post-treatment biological sample; and calculating the ratios of the levels (post-/pre-treatment) of the proteins. An increased ratio, as compared to a control, of the concentration of the proteins in the biological samples is predictive that the subject will respond to a combination therapy comprising a lenvatinib compound and a PD-1 antagonist, and a reduced ratio, as compared to a control, of the level of the proteins in the biological samples is predictive that the subject will respond less effectively to the therapy comprising a lenvatinib compound and a PD-1 antagonist than a subject having an increased ratio, as compared to a control, of the level of the protein in the biological samples.

In another embodiment, the post-treatment biological sample is obtained from the subject 1 week to 24 months after the initiation of the therapy comprising a lenvatinib compound and a PD-1 antagonist. In another embodiment, the post-treatment biological sample can be obtained from the subject 1 week to about 18 months after the initiation of the therapy comprising a lenvatinib compound and a PD-1 antagonist. In one embodiment, the post-treatment biological sample can be obtained from the subject 1 week to about 12 months after the initiation of the therapy comprising a lenvatinib compound and a PD-1 antagonist. In another embodiment, the post-treatment biological sample is obtained from the subject about 2 weeks (or 8, 9, 10, 11, 12, 13, and 14 days) to 12 months after the initiation of the therapy comprising a lenvatinib compound and a PD-1 antagonist. In another embodiment, the post-treatment biological sample can be obtained from the subject 2 weeks to 6 months after the initiation of the therapy comprising a lenvatinib compound and a PD-1 antagonist. In a further embodiment, the post-treatment biological sample can be obtained from the subject 2 weeks to 4 weeks after the initiation of the therapy comprising a lenvatinib compound and a PD-1 antagonist.

In another aspect, the disclosure provides a method of treating at least one cancer selected from the group consisting of: an endometrial cancer, a non-small cell lung cancer (NSCLC), a renal cell carcinoma ((RCC), e.g. clear cell RCC, non-clear cell RCC), a urothelial cancer, a head and neck cancer (e.g. head and neck squamous cell cancer), a melanoma (e.g., advanced melanoma such as Stage III-IV high-risk melanoma, unresectable or metastatic melanoma), a hepatocellular carcinoma, a breast cancer (e.g., triple negative breast cancer, ER⁺/HER2⁻ breast cancer), an ovarian cancer, a gastric cancer (e.g. metastatic gastric cancer or gastroesophageal junction adenocarcinoma), a colorectal cancer, a bladder cancer, a glioblastoma, a biliary tract cancer, a glioma (e.g., recurrent malignant glioma with a hypermutator phenotype), Merkel cell carcinoma (e.g., advanced or metastatic Merkel cell cancer), Hodgkin lymphoma, non-Hodgkin lymphoma (e.g. primary mediastinal B-cell lymphoma (PMBCL)), a cervical cancer, an advanced or refractory solid tumor, a small cell lung cancer (e.g., stage IV non-small cell lung cancer), a non-squamous non-small cell lung cancer, desmoplastic melanoma, a pediatric advanced solid tumor or lymphoma, a mesothelin-positive pleural mesothelioma, an esophageal cancer, an anal cancer, a salivary cancer, a prostate cancer, a carcinoid tumor, a primitive neuroectodermal tumor (pNET), and a thyroid cancer, the method including the step of administering to a subject in need thereof an effective amount of a combination therapy comprising a lenvatinib compound and a PD-1 antagonist, wherein the subject has been identified as having the ratios of the levels of one or more proteins selected from the group consisting of: IFN-γ, IL-10, CXCL9, CXCL10, CXCL11, CXCL12, FGF-19, and FGF-23 that are associated with responsiveness to this therapy.

For any of the embodiments, the biological sample can be a blood sample or any of the biological samples defined herein as long as the biological sample is consistent as between the pre-treatment sample and the post-treatment sample.

The following embodiments are envisaged for all of the above aspects. The combination therapy can use the lenvatinib or a pharmaceutically acceptable salt thereof, wherein one exemplary pharmaceutically acceptable salt is lenvatinib mesylate. The PD-1 antagonist can be selected from the group consisting of pembrolizumab, nivolumab, IBI-308, cemiplimab, JS-001, spartalizumab, SHR-1210, BGB-A317, BCD-100, durvalumab, and avelumab. In one embodiment, the PD-1 antagonist is an antagonist of PD-1. In one embodiment, the antagonist of PD-1 can be pembrolizumab or nivolumab. In one embodiment, the antagonist of PD-1 is pembrolizumab.

In particular embodiments, the PD-1 antagonist is an anti-PD-1 antibody, or antigen binding fragment thereof. In alternative embodiments, the PD-1 antagonist is an anti-PD-L1 antibody, or antigen binding fragment thereof. In some embodiments, the PD-1 antagonist can be pembrolizumab (KEYTRUDA™, Merck & Co., Inc., Kenilworth, N.J., USA), nivolumab (OPDIVO™, Bristol-Myers Squibb Company, Princeton, N.J., USA), cemiplimab (LIBTAYO™, Regeneron Pharmaceuticals, Inc., Tarrytown , N.Y., USA) durvalumab (IMFINZI™, AstraZeneca Pharmaceuticals LP, Wilmington, Del.), or avelumab (BAVENCIO™, Merck KGaA, Darmstadt, Germany)

The cancer can be endometrial cancer, non-small cell lung cancer, renal cell carcinoma, urothelial cancer, head and neck cancer, or melanoma. In one embodiment, the cancer can be an endometrial cancer. The cancer can be an advanced endometrial cancer.

In one embodiment, the proteins are one or more proteins selected from the group consisting of IFN-γ, CXCL9, CXCL10, and CXCL11. In one embodiment, the proteins are CXCL9 and/or CXCL10. In one embodiment, the proteins are FGF-19 and/or FGF-23. In one embodiment, the protein is FGF-23.

In certain embodiments, the subject is a human The biological sample can be selected from the group consisting of: a blood sample, circulating tumor cells, a plasma sample, a serum sample, a urine sample, a tissue sample, and a tumor sample. The biological sample can be a biopsy or a liquid biological sample. Liquid biological samples can be aspirates, lavages, blood, and urine, for example.

The method can further include communicating the test results to the subject's health care provider. The method further may include modifying the subject's medical record to indicate that the subject is likely or not likely to respond to a combination therapy comprising a lenvatinib compound and a PD-1 antagonist. In specific embodiments, the record can be created on a computer readable medium. In certain embodiments, the method further includes prescribing a combination therapy comprising a lenvatinib compound and a PD-1 antagonist for the subject if the ratios of the levels of the proteins are predictive that the subject will respond to a combination therapy comprising a lenvatinib compound and a PD-1 antagonist. In some embodiments, the method further includes administering to the subject a combination therapy comprising a lenvatinib compound and a PD-1 antagonist. In some embodiments, the method further includes continuing the combination therapy comprising a lenvatinib compound and a PD-1 antagonist. In some embodiments, the method further comprises selecting a subject having, or at risk of developing, a cancer that would benefit from treatment comprising a lenvatinib compound and a PD-1 antagonist.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described herein. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 displays the change in serum biomarkers level in patients as determined from serum samples obtained prior to combination therapy and after treatment, wherein the patients were treated with lenvatinib and pembrolizumab. Data are shown as median ratios (post-treatment/pre-treatment) of the serum biomarker levels with P<0.05 by the Wilcoxon signed rank test. FIG. 1A displays the ratios for Cycle 1 Day 15. FIG. 1B displays the ratios for Cycle 2 Day 1.

FIG. 2 displays the associations of change in levels of serum biomarkers with responses at cycle 2, day 1 for IL-10 (FIG. 2A), CXCL9 (FIG. 2B), CXCL10 (FIG. 2C) and CXCL12 (FIG. 2D). CR/PR/uPR is patients with complete response (CR), partial response (PR), or unconfirmed partial response (uPR). The comparative SD/PD is to patients with stable disease (SD) or progressive disease (PD). Resumed data from samples, which are not shown as dots in the range for each box plot, are shown as a number above each panel.

FIG. 3 displays the change in serum biomarkers level in patients as determined from serum samples obtained prior to combination therapy and after treatment, wherein the patients were treated with lenvatinib and pembrolizumab, in which the results of Examples 1 and 2 were integrated. Data are shown as median ratios (post-treatment/pre-treatment) of the serum biomarker levels with P<0.05 by the Wilcoxon signed rank test. FIG. 3A displays the ratios for Cycle 1 Day 15. FIG. 3B displays the ratios for Cycle 2 Day 1.

FIG. 4 displays the associations of change in levels of serum biomarkers with responses at Cycle 2, day 1 for FGF-23. CR/PR/uPR is patients with complete response (CR), partial response (PR), or unconfirmed partial response (uPR). The comparative SD/PD is to patients with stable disease (SD) or progressive disease (PD).

### DETAILED DESCRIPTION

This disclosure provides methods and compositions for predicting the response of a subject (such as a human patient) of at least one cancer selected from the group consisting of: an endometrial cancer, a non-small cell lung cancer (NSCLC), a renal cell carcinoma ((RCC), e.g. clear cell RCC, non-clear cell RCC), a urothelial cancer, a head and neck cancer (e.g. head and neck squamous cell cancer), a melanoma (e.g., advanced melanoma such as Stage III-IV high-risk melanoma, unresectable or metastatic melanoma), a bladder cancer, a hepatocellular carcinoma, a breast cancer (e.g., triple negative breast cancer, ER⁺/HER2⁻ breast cancer), an ovarian cancer, a gastric cancer (e.g. metastatic gastric cancer or gastroesophageal junction adenocarcinoma), a colorectal cancer, a glioblastoma, a biliary tract cancer, a glioma (e.g., recurrent malignant glioma with a hypermutator phenotype), Merkel cell carcinoma (e.g., advanced or metastatic Merkel cell cancer), Hodgkin lymphoma, non-Hodgkin lymphoma (e.g. primary mediastinal B-cell lymphoma (PMBCL)), a cervical cancer, an advanced or refractory solid tumor, a small cell lung cancer (e.g., stage IV non-small cell lung cancer), a non-squamous non-small cell lung cancer, desmoplastic melanoma, a pediatric advanced solid tumor or lymphoma, a mesothelin-positive pleural mesothelioma, an esophageal cancer, an anal cancer, a salivary cancer, a prostate cancer, a carcinoid tumor, a primitive neuroectodermal tumor (pNET), and a thyroid cancer to a combination therapy comprising a lenvatinib compound and a PD-1 antagonist. Another exemplary list of cancers for use with the methods described herein include a cancer selected from: melanoma, NSCLC, head and neck cancer, Hodgkin lymphoma, PMBCL, urothelial carcinoma, gastric cancer, cervical cancer, hepatocellular carcinoma, Merkel cell carcinoma, thyroid cancer, and endometrial cancer. The disclosure provides predictive biomarkers (e.g., ratios of proteins levels) to identify those subjects having, suspected of having, or at risk of developing the cancer, for whom administering a combination therapy comprising a lenvatinib compound and a PD-1 antagonist is likely to be effective or ineffective. In addition, the disclosure provides continued treatment of cancer selected from the group consisting of endometrial cancer, non-small cell lung cancer, renal cell carcinoma, urothelial cancer, head and neck cancer, melanoma, bladder cancer, hepatocellular carcinoma, breast cancer, ovarian cancer, gastric cancer, colorectal cancer, glioblastoma biliary tract cancer, and thyroid cancer with a combination therapy comprising a lenvatinib compound and a PD-1 antagonist in the subjects who are/have been predicted as responsive to the combination therapy. The biomarkers, compositions, and methods described herein are useful in selecting appropriate therapeutic modalities (e.g., a lenvatinib compound and a PD-1 antagonist therapy) for subjects suffering from at least one cancer selected from the group consisting of endometrial cancer, non-small cell lung cancer, renal cell carcinoma, urothelial cancer, head and neck cancer, melanoma, bladder cancer, hepatocellular carcinoma, breast cancer, ovarian cancer, gastric cancer, colorectal cancer, glioblastoma and biliary tract cancer, and thyroid cancer. Methods are provided for selecting patients having, suspected of having, or at risk of developing, at least one cancer selected from the group consisting of endometrial cancer, non-small cell lung cancer, renal cell carcinoma, urothelial cancer, head and neck cancer, melanoma, bladder cancer, hepatocellular carcinoma, breast cancer, ovarian cancer, gastric cancer, colorectal cancer, glioblastoma, biliary tract cancer, and thyroid cancer that could benefit from a combination therapy comprising a lenvatinib compound and a PD-1 antagonist as well as methods of treatment.

### ABBREVIATIONS

Throughout the detailed description and examples the following abbreviations will be used:
BOR Best overall response
CB Clinical benefit
CBR Clinical Benefit Rate
CR Complete Response
CXCL9 C-X-C motif chemokine ligand 9, also known as monokine induced by interferon gamma (IFN-γ), gamma-interferon-induced monokine, small-inducible cytokine B9, and C-X-C Motif Chemokine 9
CXCL10 C-X-C motif chemokine ligand 10, also known as Small inducible Cytokine Subfamily B (Cys-X-Cys), Member 10, 10 KDa Interferon Gamma-Induced Protein, SCYB10, INP10, IP10, and interferon- inducible cytokine IP-10
CXCL11 C-X-C motif chemokine ligand 11, also known as Interferon-Inducible T-Cell Alpha Chemoattractant, Interferon GammaInducible Protein 9, Beta-RI, SCYB11, SCYB9B, ITAC, and H174
CXCL12 C-X-C motif chemokine ligand 12, also known as pre-B cell growth-stimulating factor, intercrine reduced in hepatomas, stromal cell-derived factor 1, SDF1, PBSF, and IRH
DCR Disease Control Rate
DFS Disease free survival
DLT Dose limiting toxicity
DOR Duration of Response
DCR Disease Control Rate
DSDR Durable Stable Disease Rate
EDTA ethylenediamine tetraacetic acid
EGTA ethylene glycol bis(P-aminoethyl ether) N,N,N1,N1-tetraacetic acid
ELISA Enzyme-Linked Immunosorbent Assay
IFN-γ Interferon gamma
IHC Immunohistochemistry or immunohistochemical
IL-10 Interleukin 10
FGF Fibroblast growth factor
FGFR Fibroblast growth factor receptor
FR framework region
IP-10 see CXCL10
irRC Immune related response criteria
irRECIST Immune related Response Evaluation Criteria in Solid Tumors
mAb monoclonal antibody
MTD Maximum tolerated dose
NCBI National Center for Biotechnology Information
NCI National Cancer Institute
OR Overall response
ORR Objective response rate
OS Overall survival
PD Progressive disease
PDGFR Platelet-derived growth factor receptor
PD-1 Programmed Cell Death 1
PD-L1 Programmed Cell Death 1 Ligand 1, also known as B7-H1
PD-L2 Programmed Cell Death 1 Ligand 2, also known as B7-DC
PFS Progression free survival
PMSF phenylmethylsulfonyl fluoride
PR Partial response
RECIST Response Evaluation Criteria in Solid Tumors
SD Stable disease
SD Standard of Deviation—the usage of the acronym will make clear which meaning is intended.
TR Tumor response
TS Tumor shrinkage
TTR Time-to-Response
VEGFR1-3 Vascular endothelial growth factor receptor 1 to 3
uCR Unconfirmed Complete Response
uPR Unconfirmed Partial Response

### DEFINITIONS

So that the methods, compositions, and uses may be more readily understood, certain technical and scientific terms are specifically defined below. Unless specifically defined elsewhere in this document, all other technical and scientific terms used herein have the meaning commonly understood by one of ordinary skill in the art.

“About” when used to modify a numerically defined parameter (e.g., the dose of a PD-1 antagonist or a lenvatinib compound, or the length of treatment time with a combination therapy described herein) means that the parameter may vary by as much as 10% below or above the stated numerical value for that parameter. For example, a dose of “about 20 mg” may vary between 18 mg and 22 mg.

“Preferably” means a more desirable choice. For example, when used to modify a numerically defined parameter it indicates that the preferred parameter provides an improved result over another value for the parameter. This meaning of “preferably” only applies outside of the United States.

As used herein, including the appended claims, the singular forms of words such as “a,” “an,” and “the,” include their corresponding plural references unless the context clearly dictates otherwise.

“Administration” and “treatment,” as applied to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell.

“Treat” or “treating” a cancer as used herein means to administer a combination therapy of a PD-1 antagonist and a lenvatinib compound to a subject having a cancer, or diagnosed with a cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50: 1S-10S (2009)). For example, with respect to tumor growth inhibition, according to NCI standards, a T/C≥42% is the minimum level of anti-tumor activity. A T/C<10% is considered a high anti-tumor activity level, with T/C (%) =Median tumor volume of the treated/Median tumor volume of the control x 100. In some instances, response to a combination therapy described herein can be assessed using RECIST 1.1 criteria, irRC (bidimensional or unidimensional) or irRECIST criteria and the treatment achieved by a combination of a lenvatinib compound and a PD-1 antagonist can be any of PR, CR, OR, PFS, DFS and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some instances, response to a combination of a lenvatinib compound and a PD-1 antagonist can be any of PR, CR, PFS, DFS, OR and OS that is assessed using irRECIST response criteria. The treatment regimen for the disclosed combination that is effective to treat a cancer patient may vary according to factors such as the disease state, age, and weight of the patient, and the ability of the therapy to elicit an anti-cancer response in the subject. The treatment methods, medicaments, and disclosed uses may not be effective in achieving a positive therapeutic effect in every subject, they should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test, and the Wilcoxon-test.

The term “circulating tumor cells” (CTCs) refers to cells that have detached from a primary tumor and circulate in the bloodstream. CTCs may constitute seeds for subsequent growth of additional tumors (metastasis) in different tissues (Kitago et al., Clin. Chem., 55(4): 757:764 (2009)).

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the disclosed treatment methods, medicaments, and disclosed uses, unless the context requires otherwise due to express language or necessary implication.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

The terms “decreased expression ratio” and “reduced expression ratio” means a ratio of measured biomarker protein(s) that is lower than the ratio of a control protein. The term “increased expression ratio” and “elevated expression ratio” means a ratio of measured biomarkers protein(s) that is higher than the ratio of a control protein.

The term “a protein level” or “a level of a protein” refers to an amount of expression of the protein determined by a relative or absolute measuring method.

The term “lenvatinib” refers to 4-(3-chloro-4(cyclopropylaminocarbonyffaminophenoxy)-7-methoxy-6-quinolinecarboxamide

This compound is disclosed in Example 368 (see, column 270) of U.S. Pat. No. 7,253,286. U.S. Pat. No. 7,253,286 is incorporated by reference in its entirety herein. Lenvatinib mesylate is also referred to as E7080. Lenvatinib mesylate is a lenvatinib compound as the compound having the following structure:

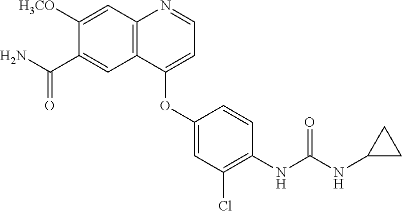 [SMILES: COc1cc2nccc(Oc3ccc(NC(=O)NC4CC4)c(Cl)c3)c2cc1C(N)=O]

The term “a PD-1 antagonist” is an agent that specifically binds to PD-1 or PD-L1 to inhibit the binding of PD-L1 to PD-1. A PD-1 antagonist can include a compound that binds to PD-1 and prevents it from binding to PD-L1. A PD-1 can also be a compound that binds to PD-L1 that prevents it from binding to PD-1. The PD-1 antagonist can be an antibody, e.g. a monoclonal antibody, or antigen-binding fragment thereof, to PD-1 or PD-L1. The term “antagonist of PD-1” can be a PD-1 antagonist that specifically binds to PD-1. The term “antagonist of PD-L1” can be a PD-1 antagonist that specifically binds to PD-L1. Non-limiting examples of an antagonist of PD-1 include: pembrolizumab, nivolumab, IBI-308, cemiplimab, JS-001, spartalizumab, SHR-1210, BGB-A317, and BCD-100. Non-limiting examples of an antagonist of PD-L1 include: durvalumab, and avelumab, but does not include atezolizumab (TECENTRIQ™, Genentech, San Francisco, Calif., USA), or CS-1001 (CStone Pharma). When a specific biologic name is referring to herein, it also can include its biosimilar as well as the reference product biologic. As used herein, a diagnostic anti-human PD-L1 mAb or an anti-hPD-L1 mAb refers to a monoclonal antibody that specifically binds to mature human PD-L1.

“Biosimilar” is a biotherapeutic product that is similar in terms of quality, safety, and efficacy to an already licensed reference biotherapeutic product, for example, defined in WHO guidelines (Guidelines on evaluation of similar Biotherapeutic Products (SBPs), Annex 2, Technical Report Series No. 977, 2009) or a biosimilar of a reference product as set forth under Biologics Price Competition and Innovation Act of 2009 and section 351(k) of the Public Health Service Act. A biosimilar has the same polypeptide sequence as the reference product. A “pembrolizumab biosimilar” means a biological product manufactured by an entity other than Merck & Co., Inc. (Kenilworth, N.J., USA) or its subsidiaries or affiliates, such as Merck Sharp & Dohme Corp. and that is approved by a regulatory agency in any country for marketing as a pembrolizumab biosimilar. A pembrolizumab biosimilar may include as the drug substance a pembrolizumab variant or an antibody with the same amino acid sequence as pembrolizumab.

As used herein, a “pembrolizumab variant” means a monoclonal antibody which comprises heavy chain and light chain sequences that are identical to those in pembrolizumab, except for having three, two or one conservative amino acid substitutions at positions that are located outside of the light chain CDRs (complementarity determining regions) and six, five, four, three, two or one conservative amino acid substitutions that are located outside of the heavy chain CDRs, e.g., the variant positions are located in the frame work regions (FR) and/or the constant region. In other words, pembrolizumab and a pembrolizumab variant comprise identical CDR sequences, but differ from each other due to having a conservative amino acid substitution at no more than three or six other positions in their full-length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

The term “pharmaceutically acceptable salt” is not particularly restricted as to the type of salt. Examples of such salts include, but are not limited to, inorganic acid addition salt such as hydrochloric acid salt, sulfuric acid salt, carbonic acid salt, bicarbonate salt, hydrobromic acid salt and hydroiodic acid salt; organic carboxylic acid addition salt such as acetic acid salt, maleic acid salt, lactic acid salt, tartaric acid salt and trifluoroacetic acid salt; organic sulfonic acid addition salt such as methanesulfonic acid salt, hydroxymethanesulfonic acid salt, hydroxyethanesulfonic acid salt, benzenesulfonic acid salt, toluenesulfonic acid salt and taurine salt; amine addition salt such as trimethylamine salt, triethylamine salt, pyridine salt, procaine salt, picoline salt, dicyclohexylamine salt, N,N′-dibenzylethylenediamine salt, N-methylglucamine salt, diethanolamine salt, triethanolamine salt, tris(hydroxymethylamino)methane salt and phenethylbenzylamine salt; and amino acid addition salt such as arginine salt, lysine salt, serine salt, glycine salt, aspartic acid salt, and glutamic acid salt. The pharmaceutically acceptable salt can be a methanesulfonic acid salt (“mesylate”). The methanesulfonic acid salt form (i.e., the mesylate) of 4-(3-chloro-4-(cyclopropylaminocarbonylaminophenoxy)-7-methoxy-6-quinolinecarboxamide is disclosed in U.S. Pat. No. 7,612,208, which is incorporated by reference herein in its entirety.

The terms “responds,” “responsive to a therapy” means that the subject administered with the therapy shows a positive response to the therapy provided. Non-limiting examples of such a positive response are: a decrease in tumor size, a decrease in metastasis of a tumor, or an increased period of survival after treatment. “Responder subject” or “responder patient” when referring to a specific anti-tumor response to treatment with a combination therapy described herein, means the subject exhibited the anti-tumor response.

“Non-responder subject” or “non-responder patient”, when referring to a specific anti-tumor response to treatment with a combination therapy described herein, means the subject did not exhibit the anti-tumor response.

“Anti-tumor response” when referring to a cancer patient treated with a therapeutic regimen, such as a combination therapy described herein, means at least one positive therapeutic effect, such as for example, reduced number of cancer cells, reduced tumor size, reduced rate of cancer cell infiltration into peripheral organs, reduced rate of tumor metastasis or tumor growth, or progression free survival. Positive therapeutic effects in cancer can be measured in a number of ways (See, W. A. Weber, J. Null. Med. 50: 1S-10S (2009); Eisenhauer et al., supra). In some instances, an anti-tumor response to a combination therapy described herein can be assessed using RECIST 1.1 criteria (response evaluation criteria in solid tumors), bidimensional irRC (immune related response criteria), or unidimensional irRC. In some instances, an anti-tumor response can be any of SD, PR, CR, PFS, or DFS.

The term “subject” or “patient” means a mammal, including but not limited to, a human, a chimpanzee, an orangutan, a gorilla, a baboon, a monkey, a mouse, a rat, a pig, a horse, a dog, and a cow.

“Bidimensional irRC” refers to the set of criteria described in Wolchok J D, et al. “Guidelines for the evaluation of immune therapy activity in solid tumors: immune-related response criteria,” Clin. Cancer Res. 2009; 15(23): 7412-7420. These criteria utilize bidimensional tumor measurements of target lesions, which are obtained by multiplying the longest diameter and the longest perpendicular diameter (cm²) of each lesion.

“CBR” or “Clinical Benefit Rate” means CR+PR+durable SD.

“DCR” or “Disease Control Rate” means CR+PR+SD.

“ORR” or “objective response rate” refers in some instances to CR+PR, and ORR_{(week 24)} refers to CR and PR measured using irRECIST in each patient in a cohort after 24 weeks of treatment with lenvatinib mesylate in combination with pembrolizumab.

“irRECIST Response Criteria” as used herein means the definition set forth in O. Bohnsack, et al., “Adaptation of the immune related response criteria: irRECIST,” Ann. Oncol. 25(Supple 4): iv361-372, 2014.

“RECIST 1.1 Response Criteria” as used herein means the definitions set forth in E.A. Eisenhauer, et al., Eur. J Cancer 45: 228-247 (2009) for target lesions or non-target lesions, as appropriate based on the context in which response is being measured.

“Sustained response” means a sustained therapeutic effect after cessation of treatment with a therapeutic agent, or a combination therapy described herein. In some instances, the sustained response has a duration that can be at least the same as the treatment duration, or at least 1.5, 2.0, 2.5 or 3 times longer than the treatment duration.

“Tissue section” refers to a single part or piece of a tissue sample, e.g., a thin slice of tissue cut from a sample of a normal tissue or of a tumor.

The terms “treatment regimen”, “dosing protocol” and “dosing regimen” are used interchangeably to refer to the dose and timing of administration of each therapeutic agent, wherein the therapeutic agents are a combination of a lenvatinib compound and a PD-1 antagonist.

By “cycle” is meant for an administration plan unit that gets repeated in the combination therapy treatment regimen. In exemplary embodiments, a cycle is a period of 3 weeks (21 days). In further exemplary embodiments, a cycle is a period of 6 weeks. “Cycle 1” refers to administration a plan unit starting from the first day of the combination therapy. “Cycle 2” refers to the cycle following cycle 1.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood or heme cancers) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor, which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Unidimensional irRC refers to the set of criteria described in M. Nishino, et al., “Developing a Common Language for Tumor Response to Immunotherapy Immune-related Response Criteria using Unidimensional measurements,” Clin. Cancer Res. 2013, 19(14): 3936-3943. These criteria utilize the longest diameter (cm) of each lesion.

IFN-γ, IL-10, CXCL9, CXCL10, CXCL11, CXCL12, FGF-19, and FGF-23 as a Biomarker for Responsiveness to the Combination Therapy

IFN-γ, IL-10, CXCL9, CXCL10, CXCL11, CXCL12, FGF-19, and FGF-23 can also be used as an effective biomarker. The ratios of the levels of one or more proteins selected from the group consisting of IFN-y, IL-10, CXCL9, CXCL10, CXCL11, CXCL12, FGF-19, and FGF-23 can be used to determine whether a subject of cancer selected from the group consisting of endometrial cancer, non-small cell lung cancer, renal cell carcinoma, urothelial cancer, head and neck cancer, melanoma, bladder cancer, hepatocellular carcinoma, breast cancer, ovarian cancer, gastric cancer, colorectal cancer, glioblastoma, biliary tract cancer, and thyroid cancer will be more likely or less likely to respond to a combination therapy comprising a lenvatinib compound and a PD-1 antagonist. In addition, the ratios of one or more proteins selected from the group consisting of IFN-γ, IL-10, CXCL9, CXCL10, CXCL11, CXCL12, FGF-19, and FGF-23 can also be used to assess or evaluate whether a subject already being administered a combination therapy comprising a lenvatinib compound and a PD-1 antagonist should continue or terminate the combination therapy.

To assess whether a subject will respond effectively to a combination therapy comprising a lenvatinib compound and a PD-1 antagonist or to evaluate continued treatment with this therapy the following method can be employed. A biological sample (e.g., blood, serum, or plasma sample) is obtained from the subject both prior to and after administration of lenvatinib compound and a PD-1 antagonist. The ratios of the levels of one or more proteins selected from the group consisting of: IFN-γ, IL-10, CXCL9, CXCL10, CXCL11, CXCL12, FGF-19, and FGF-23 in the two biological samples (level of a protein after administration of lenvatinib compound and a PD-1 antagonist/level of the protein before administration of lenvatinib compound and a PD-1 antagonist) are calculated. If the ratio of the samples from the test subject is greater than the control, the subject is determined to be likely to respond to the combination of the lenvatinib compound and a PD-1 antagonist, whereas if the ratio of the samples from the test subject is less than or about the same as (at least 90% but less than 100% of) that of the control, the subject is determined to be less likely to respond to the combination of lenvatinib compound and a PD-1 antagonist. If the subject is determined to be treatment responsive, the combination therapy with lenvatinib compound and a PD-1 antagonist continued or at least recommended to be continued. In the context of the above assay, the term “control” means samples obtained pre- and post-treatment with lenvatinib compound and a PD-1 antagonist from the same source (e.g., blood, serum or plasma sample) as that of the test samples and that are taken at the same, or substantially the same, time points from a control subject(s) as the test samples, from a subject (or subjects) who has or has not responded to treatment with lenvatinib compound and a PD-1 antagonist. The term “control” includes samples obtained in the past (pre- and post-treatment with the combination therapy) and used for future comparisons to test samples taken from subjects for which therapeutic responsiveness is to be predicted. For example, the “control” may be pre-established by an analysis of the pre- and post-treatment levels of one or more proteins selected from the group consisting of: IFN-γ, IL-10, CXCL9, CXCL10, CXCL11, CXCL12, FGF-19, and FGF-23 in one or more subjects that have or have not responded to the treatment with lenvatinib compound and a PD-1 antagonist. This pre-established ratio (which may be an average or median ratio taken from multiple subjects that have or have not responded to the therapy) may then be used for the “control” ratio in the comparison with the test sample. Non-limiting examples of such control ratio are 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9 and 4.0.

In the above method, the pre-treatment biological sample can be taken at any time point prior to treatment with the combination therapy lenvatinib compound and a PD-1 antagonist. For example, the pre-treatment biological sample may be taken minutes, hours, days, weeks, or months before initiation of the therapy, or substantially at the same time as the initiation of the therapy. The post-treatment biological sample can also be taken from the subject at any time point after initiation of treatment with the combination therapy lenvatinib compound and a PD-1 antagonist. For example, the post-treatment biological sample can be taken minutes, hours, days, weeks, or months after treatment with the combination therapy lenvatinib compound and a PD-1 antagonist. Non-limiting examples of the time points when the post-treatment biological sample is taken includes but is not limited to: 1 week to 24 months after, 1 week to 18 months after, 1 week to 12 months after, 1 week to 9 months after, 1 week to 6 months after, 1 week to 3 months after, 1 week to 9 weeks after, 1 week to 8 weeks after, 1 week to 6 weeks after, 1 week to 4 weeks after, 1 week to 2 weeks after initiation of treatment with the combination therapy lenvatinib compound and a PD-1 antagonist. The time points when the post-treatment biological sample can be taken is determined based on the cycle of the combination therapy. Non-limiting examples of such time points are: after 1st, 2nd, 3rd, 4th, 5th, 6th, 7th, 8th, 9th, 10th, 12th, 16th, 18th, 20th, 24th, 30th, and 32nd cycles.

A subject having been diagnosed with cancer can be determined to respond to a combination therapy comprising lenvatinib compound and a PD-1 antagonist, if the subject shows a partial response post treatment with the therapy. “Partial Response” means at least 30% decrease in the sum of the longest diameter (LD) of target lesions, taking as reference the baseline summed LD. A subject also can be determined to respond to a combination therapy comprising lenvatinib compound and a PD-1 antagonist, if the subject shows tumor shrinkage post-treatment with the therapy. “Tumor shrinkage” (TS) means percent change of sum of diameters of target lesions, taking as reference the baseline sum diameters. A subject can be determined to respond to a combination therapy comprising lenvatinib compound and a PD-1 antagonist, if the subject shows progression free survival. “Progression Free Survival” (PFS) refers to the period from start date of treatment to the last date before entering Progressive Disease (PD) status. “PD” means at least 20% increase in the sum of the LD of target lesions, taking as reference the smallest summed LD recorded since the treatment started, or the appearance of one or more new lesions.

An increased ratio (post-treatment/pre-treatment of the combination therapy) of levels of one or more proteins selected from the group consisting of IFN-γ, IL-10, CXCL9, CXCL10, CXCL11, CXCL12, FGF-19, and FGF-23, compared to a control (e.g., pre- and post-treatment samples obtained from a subject who is not responsive to a combination therapy comprising a lenvatinib compound and a PD-1 antagonist) is predictive of a partial response to a combination therapy comprising a lenvatinib compound and a PD-1 antagonist in patients having, suspected of having, or at risk of developing cancer selected from the group consisting of endometrial cancer, non-small cell lung cancer, renal cell carcinoma, urothelial cancer, head and neck cancer, melanoma, bladder cancer, hepatocellular carcinoma, breast cancer, ovarian cancer, gastric cancer, colorectal cancer, glioblastoma, biliary tract cancer, and thyroid cancer.

### Biological Samples

Suitable biological samples for the methods described herein include any biological fluid, cell, tissue, or fraction thereof, which includes analyte biomolecules of interest e.g. protein. A biological sample can be, for example, a specimen obtained from a subject (e.g., a mammal such as a human) or can be derived from such a subject. For example, a sample can be a tissue section obtained by biopsy, or cells that are placed in or adapted to tissue culture. A biological sample can also be a biological fluid such as urine, blood, plasma, serum, saliva, semen, sputum, cerebral spinal fluid, tears, or mucus, or such a sample absorbed onto a substrate (e.g., glass, polymer, paper) as well as medically obtained aspirates and lavages containing cells, cell proteins, and the like. A biological sample can also include a thyroid tissue sample, a renal tissue sample, a tumor sample, a cell aspirate, and circulating tumor cells. In specific embodiments, the biological sample is a tumor cell(s) or a cell(s) obtained from a region of the subject suspected of containing a tumor or a pre-cancerous lesion. For example, the biological sample may be a thyroid tumor sample or a renal tumor sample. A biological sample can be further fractionated, if desired, to a fraction containing particular cell types. For example, a blood sample can be fractionated into serum or into fractions containing particular types of blood cells such as red blood cells or white blood cells (leukocytes). If desired, a sample can be a combination of samples from a subject such as a combination of a tissue and fluid sample.

The biological samples can be obtained from a subject, e.g., a subject having, suspected of having, or at risk of developing cancer selected from the group consisting of: an endometrial cancer, a non-small cell lung cancer (NSCLC), a renal cell carcinoma ((RCC), e.g. clear cell RCC, non-clear cell RCC), a urothelial cancer, a head and neck cancer (e.g. head and neck squamous cell cancer), a melanoma (e.g., advanced melanoma such as Stage III-IV high-risk melanoma, unresectable or metastatic melanoma), a bladder cancer, a hepatocellular carcinoma, a breast cancer (e.g., triple negative breast cancer, ER⁺/HER2⁻breast cancer), an ovarian cancer, a gastric cancer (e.g. metastatic gastric cancer or gastroesophageal junction adenocarcinoma), a colorectal cancer, a glioblastoma, a biliary tract cancer, a glioma (e.g., recurrent malignant glioma with a hypermutator phenotype), Merkel cell carcinoma (e.g., advanced or metastatic Merkel cell cancer), Hodgkin lymphoma, non-Hodgkin lymphoma (e.g. primary mediastinal B-cell lymphoma (PMBCL)), a cervical cancer, an advanced or refractory solid tumor, a small cell lung cancer (e.g., stage IV non-small cell lung cancer), a non-squamous non-small cell lung cancer, desmoplastic melanoma, a pediatric advanced solid tumor or lymphoma, a mesothelin-positive pleural mesothelioma, an esophageal cancer, an anal cancer, a salivary cancer, a prostate cancer, a carcinoid tumor, a primitive neuroectodermal tumor (pNET), and a thyroid cancer. Another embodiment contemplates obtaining a biological sample from a subject, e.g., a subject having, suspected of having, or at risk of developing cancer selected from the group consisting: a melanoma, a NSCLC, a head and neck cancer, a Hodgkin lymphoma, a PMBCL, a urothelial carcinoma, a gastric cancer, a cervical cancer, a hepatocellular carcinoma, a Merkel cell carcinoma, a thyroid cancer, and an endometrial cancer. The subject can have a cancer selected from the group consisting of endometrial cancer, non-small cell lung cancer, renal cell carcinoma, urothelial cancer, head and neck cancer, and melanoma. In some embodiments, the subject has an endometrial cancer. In other embodiments, the subject has an advanced endometrial cancer. Any suitable methods for obtaining the biological samples can be employed, although exemplary methods include, e.g., phlebotomy, swab (e.g., buccal swab), or fine needle aspirate biopsy procedure. Non-limiting examples of tissues susceptible to fine needle aspiration include lymph node, lung, thyroid, breast, skin, and liver. Samples can also be collected, e.g., by microdissection (e.g., laser capture microdissection (LCM) or laser microdissection (LMD)).

Methods for obtaining and/or storing biological samples from the patient that preserve the activity or integrity of molecules (e.g., proteins) in the sample are well known to those skilled in the art. For example, a biological sample can be further contacted with one or more additional agents such as appropriate buffers and/or inhibitors, including nuclease, protease and phosphatase inhibitors, which preserve or minimize changes in the molecules (e.g., proteins) in the sample. Such inhibitors include, for example, chelators such as ethylenediamine tetraacetic acid (EDTA), ethylene glycol bis(P-aminoethyl ether) N,N,N1,N1-tetraacetic acid (EGTA), protease inhibitors such as phenylmethylsulfonyl fluoride (PMSF), aprotinin, leupeptin, antipain and the like, and phosphatase inhibitors such as phosphate, sodium fluoride, vanadate and the like. Appropriate buffers and conditions for isolating molecules are well known to those skilled in the art and can be varied depending, for example, on the type of molecule in the sample to be characterized (see, for example, Ausubel et al., Current Protocols in Molecular Biology (Supplement 47), John Wiley & Sons, New York (1999); Harlow and Lane, Antibodies: A Laboratory Manual (Cold Spring Harbor Laboratory Press (1988); Harlow and Lane, Using Antibodies: A Laboratory Manual, Cold Spring Harbor Press (1999); Tietz Textbook of Clinical Chemistry, 3rd ed. Burtis and Ashwood, eds. W. B. Saunders, Philadelphia, (1999)). A sample also can be processed to eliminate or minimize the presence of interfering substances. For example, a biological sample can be fractionated or purified to remove one or more materials that are not of interest. Methods of fractionating or purifying a biological sample include, but are not limited to, chromatographic methods such as liquid chromatography, ion-exchange chromatography, size-exclusion chromatography, or affinity chromatography. For use in the methods described herein, a sample can be in a variety of physical states. For example, a sample can be a liquid or solid, can be dissolved or suspended in a liquid, can be in an emulsion or gel, or can be absorbed onto a material.

### Quantifying of Biomarkers

In one example, an immunoassay can be used for measuring the protein level. As above, an immunoassay can be performed with an antibody that bears a detection moiety (e.g., a fluorescent agent or enzyme). Proteins from a biological sample can be conjugated directly to a solid-phase matrix (e.g., a multi-well assay plate, nitrocellulose, agarose, Sepharose, encoded particles, or magnetic beads) or it can be conjugated to a first member of a specific binding pair (e.g., biotin or streptavidin) that attaches to a solid-phase matrix upon binding to a second member of the specific binding pair (e.g., streptavidin or biotin). Such attachment to a solid-phase matrix allows the proteins to be purified away from other interfering or irrelevant components of the biological sample prior to contact with the detection antibody and also allows for subsequent washing of unbound antibody. Here as above, the presence or amount of bound detectably-labeled antibody indicates the presence or amount of protein in the biological sample.

There is no particular restriction as to the form of the antibody and the present disclosure includes polyclonal antibodies, as well as monoclonal antibodies. The antiserum obtained by immunizing animals, such as rabbits with a protein of the invention, as well polyclonal and monoclonal antibodies of all classes, human antibodies, and humanized antibodies produced by genetic recombination, are also included.

Mass spectrometry based quantitation assay methods, for example, but not limited to, multiple reaction monitoring (MRM)-based approaches in combination with stable-isotope labeled internal standards, are an alternative to immunoassays for quantitative measurement of proteins. These approaches do not require the use of antibodies and so the analysis can be performed in a cost- and time-efficient manner (see, for example, Addona et al., Nat. Biotechnol., 27: 633-641, 2009; Kuzyk et al., Mol. Cell Proteomics, 8: 1860-1877, 2009; Paulovich et al., Proteomics Clin. Appl., 2: 1386-1402, 2008). In addition, MRM offers superior multiplexing capabilities, allowing for the simultaneous quantification of numerous proteins in parallel. The basic theory of these methods has been well-established and widely utilized for drug metabolism and pharmacokinetics analysis of small molecules.

Methods for measuring protein levels can optionally be performed in formats that allow for rapid preparation, processing, and analysis of multiple samples. This can be, for example, in multi-welled assay plates (e.g., 96 wells or 386 wells) or arrays (e.g., protein chips). Stock solutions for various reagents can be provided manually or robotically, and subsequent sample preparation (e.g., labeling, or cell fixation), pipetting, diluting, mixing, distribution, washing, incubating (e.g., antibody binding), sample readout, data collection (optical data) and/or analysis (computer aided image analysis) can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay. Exemplary high-throughput cell-based assays (e.g., detecting the presence or level of a target protein in a cell) can utilize ArrayScan® VTI HCS Reader or KineticScan® HCS Reader technology (Cellomics Inc., Pittsburg, Pa.).

In some embodiments, the levels of one protein, two proteins, three proteins, four proteins, five proteins, six proteins, seven proteins, or eight proteins selected from the group consisting of IFN-γ, IL-10, CXCL9, CXCL10, CXCL11, CXCL12, FGF-19 and FGF-23 can be measured and their ratios of post-treatment level/pre-treatment level can be calculated. In one embodiment, the levels of two proteins, three proteins, or four proteins is selected from the group consisting of IFN-y, CXCL9, CXCL10 and CXCL11. In one embodiment, the levels of CXCL9 and/or CXCL10 can be measured and their ratios of post-treatment level/pre-treatment level can be calculated.

A person performing the steps of the disclosed methods, such as taking biological samples, measuring the protein levels in the biological samples, and calculating the ratios of the protein levels may be same or different. A doctor may perform or have a medical practitioner performed such steps. The term “medical practitioner” refers to any health care provider, such as a doctor, a physician's assistant, a laboratory technician (including an expert for performing testing in a testing service center), a nurse and other workers of medical institutions.

### Cut-Off Values

The methods described herein can involve, assessing the ratios (post-treatment level/pre-treatment level with the drug combination) of one or more proteins selected from the group consisting of IFN-γ, IL-10, CXCL9, CXCL10, CXCL11, CXCL12, FGF-19, and FGF-23, wherein the ratio predicts the response of a subject to a combination therapy comprising a lenvatinib compound and a PD-1 antagonist. “Assessing” can include, e.g., comparing the ratio of test biological sample with a known (e.g., in a reference biological sample) or a control ratio of the particular protein(s) of interest. For example, the ratio of one or more of the proteins in a test biological sample can be compared to the corresponding ratio in a subject who has responded to or failed to respond to a combination therapy comprising lenvatinib compound and a PD-1 antagonist, or an average or median ratio of the protein level of multiple (e.g., two, three, four, five, six, seven, eight, nine, 10, 15, 20, 25, 30, 35, or 40 or more) subjects, of the same species, who have responded to or have failed to respond to the combination therapy. Assessing can also include determining if the ratios of the protein levels fall within a range of values predetermined as predictive of responsiveness of a subject to a combination therapy comprising a lenvatinib compound and a PD-1 antagonist. Assessing also can be, or include, determining if the ratios of the protein levels fall above or below a predetermined cut-off value. A cut-off value is typically a ratio of the protein levels, above or below which is considered predictive of responsiveness of a subject to a combination therapy comprising a lenvatinib compound and a PD-1 antagonist. Some cut-off values are not absolute in that clinical correlations can still remain significant over a range of values on either side of the cutoff; however, it is possible to select an optimal cut-off value (e.g., varying H-scores, also referred to as a “histo” or “histology score”) of the ratios for a particular sample types and cancer types. Cut-off values determined for use in the methods described herein can be compared with, e.g., published ranges but can be individualized to the methodology used and patient population. It is understood that improvements in optimal cut-off values could be determined depending on the sophistication of statistical methods used and on the number and source of samples used to determine reference ratios for the different proteins, sample types and cancer types. Therefore, established cut-off values can be adjusted up or down, on the basis of periodic re-evaluations or changes in methodology or population distribution.

The reference ratios of levels of one or more proteins can be determined by a variety of methods. The reference ratio can be determined by comparison of the ratio of a protein of interest in, e.g., populations of subjects (e.g., patients) that are responsive to a combination therapy comprising a lenvatinib compound and a PD-1 antagonist. This can be accomplished, for example, by histogram analysis, in which an entire cohort of patients is graphically presented, wherein a first axis represents the ratio and a second axis represents the number of subjects in the cohort whose sample contain one or more ratios at a given amount. Determination of the reference ratio can then be made based on an amount which best distinguishes these separate groups. The reference ratio can be a single number, equally applicable to every subject, or the reference ratio can vary, according to specific subpopulations of subjects. For example, older subjects can have a different reference ratio than younger subjects for the same metabolic disorder. In addition, a subject with more advanced disease (e.g., a more advanced form of a disease treatable by lenvatinib compound and a PD-1 antagonist) can have a different reference value than one with a milder form of the disease.

### Creating a Response Profile

The methods described herein can also be used to generate a response profile for a subject to the combination therapy. The profile can include information regarding the ratios of levels of one or more proteins selected from the group consisting of IFN-γ, IL-10, CXCL9, CXCL10, CXCL11, CXCL12, FGF-19, and FGF-23 in a biological sample (e.g., plasma, serum) of the subject post/pre-treatment with lenvatinib compound and a PD-1 antagonist. A response profile can include the levels, or the ratio of the levels of one or more additional proteomic markers, serum markers, or clinical markers. The markers (biomarkers) measured are protein markers.

It is understood that the response profile can be in electronic form (e.g., an electronic patient record stored on a computer or other electronic (computer-readable) media such as a DVD, CD, or floppy disk, optical disc, barcodes, or magnetic ink characters) or written form. The response profile can also include information for several (e.g., two, three, four, five, 10, 20, 30, 50, or 100 or more) subjects (e.g., human patients). Such multi-subject response profiles can be used, e.g., in analyses (e.g., statistical analyses) of particular characteristics of subject cohorts.

Responsiveness of a subject to a combination therapy comprising a lenvatinib compound and a PD-1 antagonist can be classified in several ways and classification is dependent on the subject's disease, the severity of the disease, and the particular medicament the subject is administered. In the simplest sense, responsiveness is any decrease in the disease state as compared to pre-treatment, and non-responsiveness is the lack of any change in the disease state as compared to pre-treatment. Responsiveness of a subject (e.g., a human) with a cancer can be classified based on one or more of a number of objective clinical indicia such as, but not limited to, tumor size, Clinical Benefit (CB), Overall Survival (OS), Progression Free Survival (PFS), Disease Control Rate (DCR), Time-To-Response (TTR), Tumor Shrinkage (TS), or Tumor Response (TR).

“Clinical benefit” refers to having one of the following statuses—Complete Response (CR), Partial Response (PR); or Stable Disease (SD) with 6 months or more progression free survival (PFS). “Complete Response” means complete disappearance of all target lesions. “Partial Response” means at least 30% decrease in the sum of the longest diameter (LD) of target lesions, taking as reference the baseline summed LD. “Progressive Disease” (PD) means at least a 20% increase in the sum of the LD of target lesions, taking as reference the smallest summed LD recorded since the treatment started, or the appearance of one or more new lesions. “Stable Disease” means neither sufficient shrinkage of the target lesions to qualify for “PR” (partial response) nor sufficient increase to qualify for progressive disease (PD), taking as reference the smallest summed LD since the treatment started.

“Overall Survival” (OS) is defined as the time from randomization until death from any cause. “Randomization” means randomization of a patient into a test group or a control group when therapy plan for a patient is determined.

“Progression Free Survival” (PFS) refers to the period from start date of treatment to the last date before entering PD status.

“Disease Control Rate” (DCR) is defined as CR or PR or SD for 7 weeks.

“Time-To-Response” (TTR) is defined as the time from the date of initiation of treatment to the date when criteria for response (CR or PR for complete response and partial response respectively) are first met.

“Tumor shrinkage” (TS) means percent change of sum of diameters of target lesions, taking as reference the baseline sum diameters.

“Tumor response” (TR) compares subjects with “Partial Response” (PR) with subjects with either Stable Disease (SD) or Progressive Disease (PD).

### Methods of Treatment

The methods disclosed herein enable the assessment of a subject for responsiveness to a combination therapy comprising lenvatinib compound and a PD-1 antagonist. A subject who is likely to respond to the combination therapy can be administered lenvatinib compound and a PD-1 antagonist.

The methods of this disclosure also enable the classification of subjects into groups of subjects that are more likely to benefit, and groups of subjects that are less likely to benefit, from treatment with lenvatinib compound and a PD-1 antagonist. The ability to select such subjects from a pool of subjects who are being considered for treatment with lenvatinib compound and a PD-1 antagonist is beneficial for effective treatment.

The methods can also be used to determine whether to continue the combination therapy comprising lenvatinib compound and a PD-1 antagonist after administering this therapy for a short period of time and determining based on the ratios of the levels of one or more of the biomarkers described above post-treatment versus pre-treatment whether this therapy is more likely or less likely to benefit the patient.

The subject to be treated with the combination therapy has, is suspected of having, or is likely to develop cancer selected from the group consisting of endometrial cancer, non-small cell lung cancer, renal cell carcinoma, urothelial cancer, head and neck cancer, melanoma, bladder cancer, hepatocellular carcinoma, breast cancer, ovarian cancer, gastric cancer, colorectal cancer, glioblastoma and biliary tract cancer, and thyroid cancer. In one embodiment, the subject to be treated with the combination therapy has, is suspected of having, or is likely to develop cancer selected from the group consisting of endometrial cancer, non-small cell lung cancer, renal cell carcinoma, urothelial cancer, head and neck cancer, melanoma. In one embodiment, the subject to be treated with the combination therapy has, is suspected of having, or is likely to develop cancer selected from the group consisting of endometrial cancer, including advanced endometrial cancer.

If the subject is more likely to respond to a combination therapy comprising a lenvatinib compound and a PD-1 antagonist (based on ratios of the levels of biomarkers described above), the subject can then be administered an effective amount of the lenvatinib compound (e.g., lenvatinib mesylate) and a PD-1 antagonist. An effective amount of the compound and the antagonist can suitably be determined by a health care practitioner taking into account, for example, the characteristics of the patient (age, sex, weight, race, etc.), the progression of the disease, and prior exposure to the drug. The dosage regimens of the lenvatinib compound and the antagonist can be determined, for example, by combining their approved dosage regimens. Such approved dosage regimens are: 24 mg/day, 20 mg/day, 14 mg/day, 12 mg/day, or 8 mg/day administered once daily orally for lenvatinib mesylate; 200 mg administered every 3 weeks intravenously for pembrolizumab; 3 mg/kg patient weight or 240 mg administered every 2 weeks, 2 mg/kg patient weight administered every 3 weeks, or 480 mg administered every 4 weeks intravenously for nivolumab; 10 mg/kg patient weight administered every 2 weeks intravenously for durvalumab; and 10 mg/kg patient weight administered every 2 weeks intravenously for avelumab. In this case, the treatment cycle of the combination therapy can be determined as the least common multiple of the frequency of administration of each of the lenvatinib compounds and the antagonists.

In some embodiments, the PD-1 antagonist is pembrolizumab. In particular sub-embodiments, the method can comprise administering 200 mg of pembrolizumab to the patient about every three weeks. In other sub-embodiments, the method can comprise administering 400 mg of pembrolizumab to the patient about every six weeks.

In further sub-embodiments, the method can comprise administering 2 mg/kg of pembrolizumab to the patient about every three weeks. In particular sub-embodiments, the patient is a pediatric patient.

In some embodiments, the PD-1 antagonist can be nivolumab. In particular sub-embodiments, the method can comprise administering 240 mg of nivolumab to the patient about every two weeks. In other sub-embodiments, the method can comprise administering 480 mg of nivolumab to the patient about every four weeks.

In some embodiments, the PD-1 antagonist can be cemiplimab. In particular embodiments, the method can comprise administering 350 mg of cemiplimab to the patient about every 3 weeks.

In some embodiments, the PD-1 antagonist can be durvalumab. In particular sub-embodiments, the method can comprise administering 10 mg/kg of durvalumab to the patient about every two weeks.

In some embodiments, the PD-1 antagonist can be avelumab. In particular sub-embodiments, the method can comprise administering 800 mg of avelumab to the patient about every two weeks.

In some embodiments, the PD-1 antagonist can be administered intravenously (i.v. or IV). In alternative embodiments, the PD-1 antagonist can be administered subcutaneously (s.c. or SC).

If the subject is less likely to respond to a combination therapy comprising a lenvatinib compound and a PD-1 antagonist, the subject can then be optionally administered a therapy other than the combination therapy. These therapies include, but are not limited to, “standard of care” treatment (i.e., prevailing standard of care as determined by the health care practitioner or as specified in the clinical study) such as investigational drugs and chemotherapy.

Subjects of all ages can be affected by disorders treatable by the combination therapy. Therefore, a biological sample used in a methods described herein can be obtained from a subject (e.g., a human) of any age, including a child, an adolescent, or an adult, such as an adult having, or suspected of having, a disease treatable by the combination therapy.

The methods can also be applied to individuals at risk of developing a cancer treatable by the combination therapy. Such individuals include those who have (i) a family history of (a genetic predisposition for) such disorders or (ii) one or more risk factors for developing such disorders.

After classifying or selecting a subject based on whether the subject will be more likely or less likely to respond to the combination therapy, a medical practitioner (e.g., a doctor) can administer the appropriate therapeutic modality to the subject. Methods of administering the lenvatinib compound or the antagonist are well known in the art, e.g. described in their product labels.

It is understood that any therapy described herein (e.g., a combination therapy comprising a lenvatinib compound and a PD-1 antagonist or a therapy other than the combination therapy) can include one or more additional therapeutic agents. That is, any therapy described herein can be co-administered (administered in combination) with one or more additional anti-tumor agents. Furthermore, any therapy described herein can include one or more agents for treating, for example, pain, nausea, and/or one or more side-effects of a combination therapy comprising a lenvatinib compound and a PD-1 antagonist.

The combination therapies comprising a lenvatinib compound can be, e.g., simultaneous or successive. For example, lenvatinib compounds (e.g., lenvatinib mesylate) and a PD-1 antagonist can be administered at the same time or a lenvatinib compound (e.g., lenvatinib mesylate) can be administered first in time and a PD-1 antagonist administered second in time, or vice versa. The dosing frequency of the lenvatinib compound and the PD-1 antagonist can be different or same. In one embodiment, the dosing frequency is different. An exemplary dosing frequency of the lenvatinib compound can be once daily and dosing frequency of the PD-1 antagonist can be once in a few weeks, for example, 1 week, 2 weeks, 3 weeks, 4 weeks or 1 month, or 6 weeks. The dosing frequency of the lenvatinib compound can be once daily and dosing frequency of the PD-1 antagonist can be once in 2 weeks, in which one treatment cycle of the combination therapy is defined as 2 weeks. The dosing frequency of the lenvatinib compound can be once daily and dosing frequency of the PD-1 antagonist is once every 3 weeks or once every 6 weeks, in which one treatment cycle of the combination therapy is defined as 3 weeks. In one embodiment, dosing frequency of lenvatinib mesylate can be once daily and the dosing frequency of pembrolizumab is once in 3 weeks. In one embodiment, dosing frequency of the lenvatinib compound is once daily and dosing frequency of nivolumab is once in 2 weeks, 3 weeks, or 4 weeks.

In some embodiments, the PD-1 antagonist can be administered intravenously or subcutaneously.

In cases where the subject predicted to respond to a combination therapy comprising a lenvatinib compound (e.g., lenvatinib mesylate) and a PD-1 antagonist has been previously administered one or more therapies other than lenvatinib or a PD-1 antagonist, the combination therapy can replace or augment a previously or currently administered therapy. For example, upon treating with the combination therapy, administration of the one non-lenvatinib therapies can cease or diminish, e.g., be administered at lower levels. Administration of the previous therapy can be maintained while the combination therapy is administered. In some embodiments, a previous therapy can be maintained until the level of the combination therapy reaches a level sufficient to provide a therapeutic effect.

### Kits

This application also provides kits. In certain embodiments, the kits include antibodies that can be used to measure the levels of one or more proteins selected from the group consisting of IFN-γ, IL-10, CXCL9, CXCL10, CXCL11, CXCL12, FGF-19, and FGF-23 The kits can, optionally, contain instructions for measuring the levels of one or more proteins selected from the group consisting of IFN-γ, IL-10, CXCL9, CXCL10, CXCL11, CXCL12, FGF-19, and FGF-23 in a biological sample.

The kits can optionally include, e.g., a control biological sample set containing known amounts or concentrations of the one or more biomarker proteins. In some instances, the control can be with an insert (e.g., a paper insert or electronic medium such as a CD, DVD, or floppy disk) containing ratios of the levels of one or more proteins predictive of a response to a combination therapy comprising a lenvatinib compound and a PD-1 antagonist.

The kits can include one or more reagents for processing a biological sample. For example, a kit can include reagents for isolating a protein from a biological sample and/or reagents for measuring amount or concentration of a protein in a biological sample (e.g., an antibody that binds to the protein that is the subject of the detection assay and/or an antibody that binds the antibody that binds to the protein).

The kits can include a software package for analyzing the results.

The kits can also include one or more antibodies for measuring amount or concentration of one or more proteins in a biological sample, where the proteins sought for detection in the samples are selected from the group consisting of IFN-γ, IL-10, CXCL9, CXCL10, CXCL11, CXCL12, FGF-19, and FGF-23. For example, a kit can include (or in some cases consist of) a plurality of antibodies capable of specifically binding to one or more proteins selected from the group consisting of IFN-γ, IL-10, CXCL9, CXCL10, CXCL11, CXCL12, FGF-19, and FGF-23 and optionally, instructions for measuring amount or concentration of the proteins and/or a detection antibody comprising a detectably-labeled antibody that is capable of binding to at least one antibody of the plurality. The kits can include antibodies that recognize one, two, three, four, five, or six proteins selected from the group consisting of IFN-γ, IL-10, CXCL9, CXCL10, CXCL11, CXCL12, FGF-19, and FGF-23.

The kits described herein can also, optionally, include instructions for administering a combination therapy comprising a lenvatinib compound and a PD-1 antagonist, where the ratios (post-treatment/pre-treatment of the combination therapy) of the levels of the measured proteins from a subject's biological sample, wherein the proteins are selected from the group consisting of IFN-γ, IL-10, CXCL9, CXCL10, CXCL11, CXCL12, FGF-19, and FGF-23 predicts that a subject will respond to a combination therapy comprising a lenvatinib compound and a PD-1 antagonist.

### General Methods

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

Additional diagnostic methods for using biomarkers would be as described by the Clinical Laboratory Improvement Amendments (CLIA) of the US FDA which regulates laboratory testing.

The following are examples of the methods disclosed herein. They are not to be construed as limiting the scope of the claims.

### EXAMPLES

### Example 1

Biomarker Assessments in Endometrial Cancer Patients for Treatment with Lenvatinib Plus Pembrolizumab

Methods: This study is a part of the phase 1b/2 study of the combination of lenvatinib mesylate (referred to as “lenvatinib” hereafter in Examples) plus pembrolizumab in patients with selected solid tumors (Clinicaltrials.gov ID: NCT02501096). The patients permitted in the study had one of the following tumors: a non-small cell lung cancer, a renal cell carcinoma, an endometrial cancer, a urothelial cancer, a squamous cell carcinoma of the head and neck, or a melanoma.

The phase 1b part of the clinical trial aimed to determine and confirm the maximum tolerated dose (MTD) of lenvatinib mesylate in combination with pembrolizumab (200 mg intravenously [IV, intravenous] every 3 weeks). Lenvatinib was administered with water orally once a day (with or without food) continuously in the 21-day treatment cycle. Lenvatinib starting dose was 24 mg given to the patient orally once daily, and was reduced, if required to either 20 mg or 14 mg. If two or more patients at a dose level experienced a DLT (dose limiting toxicity), the trial proceeded with enrollment in the next lower dose level, with the dose reduction of lenvatinib being reduced from 24 mg to 20 mg or from 20 mg to 14 mg.

The phase 1b part of the study aimed to enroll from 10 to 30 patients with select solid tumors (i.e., a non-small cell lung cancer, a renal cell carcinoma, an endometrial cancer, a urothelial cancer, a squamous cell carcinoma of the head and neck, or a melanoma). The phase 2 expansion part of the clinical study aimed to enroll up to 20 patients in up to 6 cohorts representing the tumor types of interest enrolled in the phase 1b of the clinical study.

Eligible patients for the endometrial cancer cohort were ≥18 years of age, with confirmed endometrial carcinoma that progressed after treatment with approved therapy or for which there are no standard effective therapies available, and measurable disease according to immune-related Response Evaluation Criteria in Solid Tumors (irRECIST). Patients enrolled in the phase 2 part of the trial could have received up to 2 prior lines of systemic therapy. All eligible patients had an Eastern Cooperative Oncology Group (ECOG) performance status of 0 or 1, adequately controlled blood pressure, adequate renal, bone marrow, blood coagulation, and liver function, and life expectancy >12 weeks. Prior treatment with lenvatinib or any PD-1, anti-PD-L1, or anti-PD-L2 agent was not permitted for any patient enrolled in phase 2. Patients received oral lenvatinib 20 mg/day plus pembrolizumab 200 mg intravenously every 3 weeks on a 21-day treatment cycle.

The primary outcome measures used:
1. MTD (maximum tolerated dose) (Phase 1b) [Time Frame: Cycle 1 (21 Days)]
2. Objective response rate (ORR) at Week 24 [Time Frame: Week 24]
3. Dose Limiting Toxicity (DLT) [Time Frame: Cycle 1 (21 Days) ]

The secondary outcome measures utilized are:
(a) Number of participants with Treatment emergent adverse events (TEAEs) and treatment emergent serious adverse events (SAEs) [Time Frame: For each participant, from the first dose till 90 days after the last dose, unless participant starts new anticancer drug then 30 days, or up to approximately 2 years.]
(b) ORR [Time Frame: From date of first dose of study drug until disease progression, development of unacceptable toxicity, withdrawal of consent, or up to approximately 2 years.]
(c) Progression-free survival (PFS) [Time Frame: From the date of first dose of study drug to the date of first documentation of confirmed disease progression or death (whichever occurs first) or up to approximately 2 years.]
(d) Overall survival (OS) [Time Frame: From the date of first dose of study drug until date of death from any cause or up to approximately 2 years.]
(e) Duration of response (DOR) [Time Frame: From date of first dose of study drug until disease progression, development of unacceptable toxicity, withdrawal of consent, or up to approximately 2 years.]
(f) Disease control rate (DCR) [Time Frame: From date of first dose of study drug until disease progression, development of unacceptable toxicity, withdrawal of consent, or up to approximately 2 years.]
(g) Durable Stable Disease rate (DSDR) [Time Frame: From date of first dose of study drug until disease progression, development of unacceptable toxicity, withdrawal of consent, or up to approximately 2 years.]
(h) Clinical benefit rate (CBR) [Time Frame: From date of first dose of study drug until disease progression, development of unacceptable toxicity, withdrawal of consent, or up to approximately 2 years.]
(i) Area under the curve (AUC) of lenvatinib [Time Frame: 0.5-4 hours (hrs), and 6-10 hrs post lenvatinib dose on C1D1, pre-dose, 0.5-4 hrs, and 6-10 hrs post lenvatinib dose on C1D15, and pre-pembrolizumab dose and 2-12 hrs post lenvatinib dose on C2D1. Pre-pembrolizumab dose only on Day 1 of Cycles 3 to 6.]
(j) Apparent clearance of lenvatinib [Time Frame: 0.5-4 hours (hrs), and 6-10 hrs post lenvatinib dose on C1D1, pre-dose, 0.5-4 hrs, and 6-10 hrs post lenvatinib dose on C1D15, and pre-pembrolizumab dose and 2-12 hrs post lenvatinib dose on C2D1. Pre-pembrolizumab dose only on Day 1 of Cycles 3 to 6.]

Peripheral blood for serum preparation was collected from 42 patients before the first dose of study drug (the drug combination of lenvatinib and pembrolizumab) and before the doses of cycle 1 day 15, and cycle 2 day 1 and available serum samples in 37 patients out of 42 patients were assayed. In detail, the number of the samples available for the assay were 37 (baseline), 31 (cycle 1 day 15) and 35 (cycle 2 day 1). A total of 41 candidate serum biomarkers either associated with lenvatinib or reported for immune checkpoint inhibitors in the literature were assayed using 18 preconfigured CustomMAP immunoassay panels and measured by a multiplex flow cytometry-based platform by the manufacturer (Multi-Analyte Profile (MAP)) in addition to two (2) Quanterix Simoa assays at a Myriad RBM (Austin, Tex., USA). Serum biomarkers for which >20% of patients had out-of-range measurements were not included in correlative analyses. Post-treatment serum biomarker fold changes from baseline, and serum biomarker analyses designed to test relationships between change levels of serum biomarkers and tumor responses (CR/PR/uPR vs SD/PD) in cycle 2 day 1 were analyzed by the Wilcoxon rank sum test for the patient whose serum biomarker data and tumor response data were both available (29 patients). There was no patient with unconfirmed complete response (uCR). Results with P<0.05 with false discovery rates (FDR) for multiple comparisons are reported for the biomarker analyses. Statistical analyses were performed using SAS (SAS Institute, Inc., Cary, N.C., USA), version 9 or higher. The clinical study, uCR/uPR is considered confirmed becoming CR and PR if the clinical response is observed again no less than 4 weeks later. Said differently, the patients for whom CR/PR is observed only once are regarded as uCR/uPR, whereas patients in which the response is observed at least twice separated by a period of no less than 4 weeks are regarded as CR/PR.

Results: In this exploratory serum biomarker analysis of a limited number of patients, Panels of 41 candidate PD biomarkers, which were selected for each monotherapy based on reported clinical PD biomarker analysis for lenvatinib, and the literature information for pembrolizumab were analyzed.

TABLE 1
List of candidate PD biomarkers

| Analyte | |
|---|---|
| Short Name | Analyte Name |
| ANG-1 | Angiopoietin-1 |
| ANG-2 | Angiopoietin-2 |
| EGF | Epidermal Growth Factor |
| CCL11 | Eotaxin-1 |
| FGF-21 | Fibroblast Growth Factor 21 |
| FGF-23 | Fibroblast growth factor 23 |
| FRTN | Ferritin |
| G-CSF | Granulocyte Colony-Stimulating Factor |
| HGF | Hepatocyte Growth Factor |
| ICAM-1 | Intercellular Adhesion Molecule 1 |
| IFN-gamma | Interferon gamma, IFN-γ |
| IL-1 beta | Interleukin-1 beta, IL-1β |
| IL-10 | Interleukin-10 |
| IL-12p70 | Interleukin-12 Subunit p70 |
| IL-13 | Interleukin-13 |
| IL-18 | Interleukin-18 |
| IL-2 receptor | Interleukin-2 receptor alpha |
| alpha | |
| IL-4 | Interleukin-4 |
| IL-6 | Interleukin-6 |
| IL-8 | Interleukin-8 |
| CXCL10 | Interferon gamma Induced Protein 10 |
| CXCL11 | Interferon-inducible T-cell alpha chemoattractant |
| CCL2 | Monocyte Chemotactic Protein 1 |
| MICA | MHC class I chain-related protein A |
| CXCL9 | Monokine Induced by Gamma Interferon |
| CCL3 | Macrophage Inflammatory Protein-1 alpha |
| MMP-3 | Matrix Metalloproteinase-3 |
| PDGF-BB | Platelet-Derived Growth Factor BB |
| PLGF | Placenta Growth Factor |
| CCL5 | T-Cell-Specific Protein RANTES |
| CXCL12 | Stromal cell-derived factor-1 |
| TIE-2 | Tyrosine kinase with Ig and EGF homology domains 2 |
| TIMP-1 | Tissue Inhibitor of Metalloproteinases 1 |
| TNF-alpha | Tumor Necrosis Factor alpha |
| VCAM-1 | Vascular Cell Adhesion Molecule-1 |
| VDBP | Vitamin D-Binding Protein |
| VEGF | Vascular Endothelial Growth Factor |
| VEGFR-1 | Vascular Endothelial Growth Factor Receptor 1 |
| VEGFR-2 | Vascular Endothelial Growth Factor Receptor 2 |
| VEGFR-3 | Vascular endothelial growth factor receptor 3 |
| vWF | von Willebrand Factor |

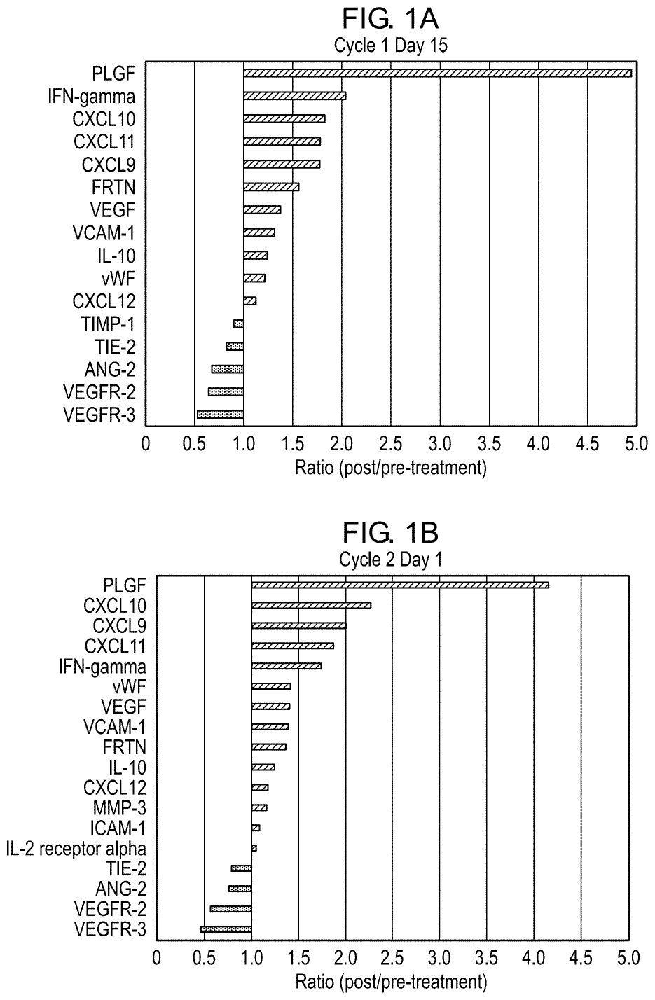

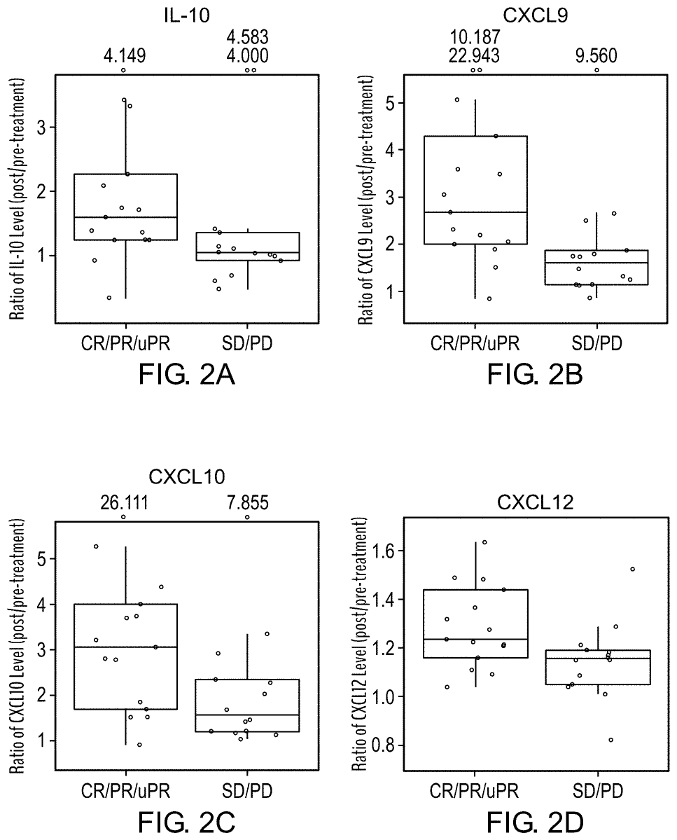

Changes levels were observed in 16 and 18 of the 41 examined serum biomarkers at post-treatments with lenvatinib and pembrolizumab at cycle 1 day 15 and cycle 2 day 1, respectively (FIG. 1, Panels A and B respectively). Among them, increased levels of interferon gamma (IFN-γ)-regulated chemokines (i.e., CXCL9, CXCL10, CXCL11) along with IFN-γ itself were observed with treatments with lenvatinib mesylate and pembrolizumab (FIG. 1) and associations were found between increases of post/pre-treatment ratio of CXCL9 and CXCL10 levels and responses with the combination therapy (FIG. 2). These results might suggest roles of activation of interferon-gamma pathway with the combination therapy with lenvatinib and pembrolizumab. In addition, associations were found between increases of post/pre-treatment ratio of IL-10 and CXCL12 levels and responses with the combination therapy (FIG. 2).

While there was a large change in PLGF as reflected in FIG. 1, the change was not found to correlate with clinical outcome.

### Example 2

### Biomarker Assessments in Endometrial Cancer Patients for Treatment with Lenvatinib Plus Pembrolizumab Using an ELISA

FGF19 and FGF-23 in the same serum samples were assayed, measured and analyzed in Example 1 in a similar manner as Example 1, where the assay using 20 preconfigured CustomMAP immunoassay panels in Example 1 were replaced with the assay using two (2) ELISA kits (R&D, Human FGF-19 Quantikine ELISA Kit (DF1900), and Kainos, FGF23 ELISA Kit (CY-4000)) according to manufacturers' instructions and using the manufacturers' provided antibodies, and the sample number in cycle 2 day 1 for FGF-19 were 34.

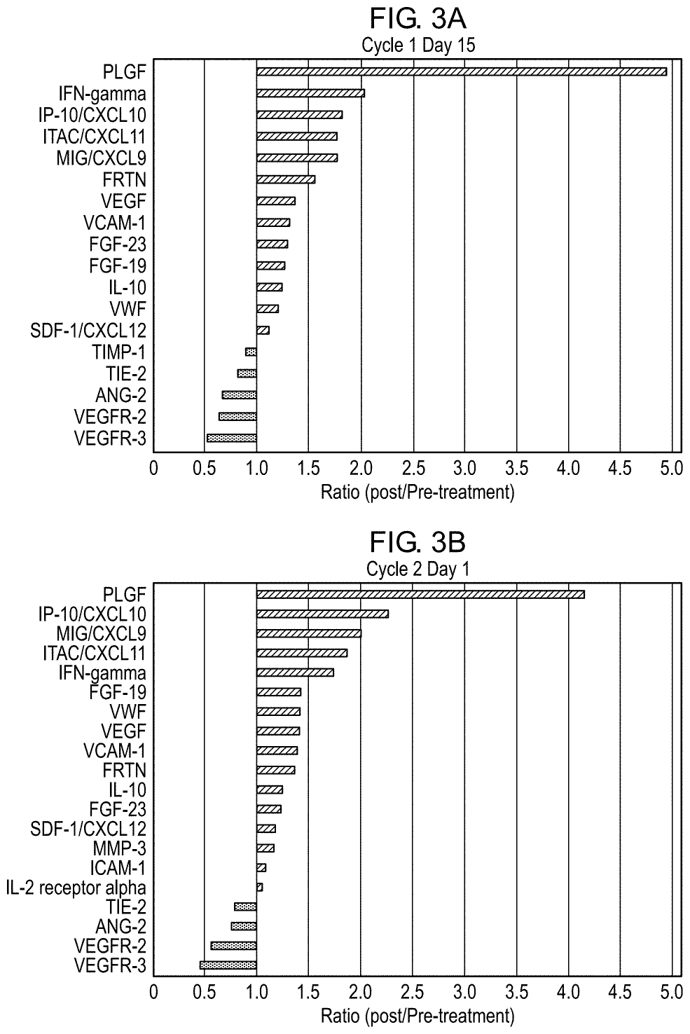

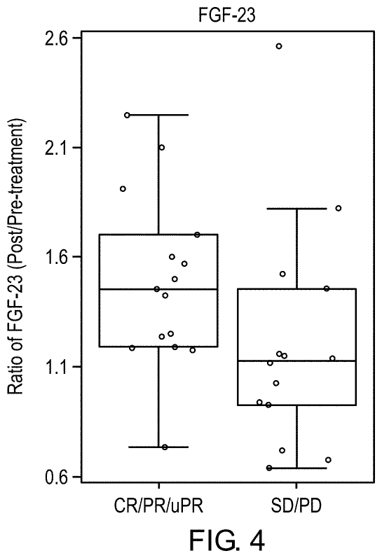

Changes levels were observed in FGF-19 (Fibroblast Growth Factor 19) and FGF-23 at post-treatments with lenvatinib and pembrolizumab at cycle 1 day 15 and cycle 2 day 1, respectively (FIG. 3, Panels A and B respectively, in which the data of Example 2 were integrated with the data of Example 1). Increased levels of FGF-23 were observed with treatments with lenvatinib mesylate and pembrolizumab (FIG. 3) and associations were found between increases of post/pre-treatment ratio of FGF-23 levels and responses with the combination therapy (FIG. 4) in the 29 subjects studied.

### Example 3

### Biomarker Assessments in Patients with the Other Solid Tumor for Treatment with Lenvatinib Plus Pembrolizumab

According to the similar manner in Example 1, biomarker assessment in the other solid tumors, such as a non-small cell lung cancer, a renal cell carcinoma, an urothelial cancer, a head and neck cancer including squamous cell carcinoma of the head and neck, a melanoma, a bladder cancer, a hepatocellular carcinoma, a breast cancer, an ovarian cancer, a gastric cancer, a colorectal cancer, a glioblastoma, a biliary tract cancer, or a thyroid cancer can be assessed. Additional cancers that may be assessed include: an endometrial cancer, a glioma (e.g., recurrent malignant glioma with a hypermutator phenotype), Merkel cell carcinoma (e.g., advanced or metastatic Merkel cell cancer), Hodgkin lymphoma, non-Hodgkin lymphoma (e.g. primary mediastinal B-cell lymphoma (PMBCL)), a cervical cancer, an advanced or refractory solid tumor, a small cell lung cancer (e.g., stage IV non-small cell lung cancer), a non-squamous non-small cell lung cancer, desmoplastic melanoma, a pediatric advanced solid tumor or lymphoma, a mesothelin-positive pleural mesothelioma, an esophageal cancer, an anal cancer, a salivary cancer, a prostate cancer, a carcinoid tumor, and a primitive neuroectodermal tumor (pNET).

### Specific Embodiments

Specific embodiments of the invention can include the following:
1. A method of predicting the response of a human subject having or suspected of having at least one cancer to a combination therapy comprising lenvatinib or a pharmaceutically acceptable salt thereof and a Programmed Cell Death 1 protein (PD-1) antagonist, the method comprising:
   (a) measuring levels of one or more proteins selected from the group consisting of: IFN-γ, IL-10, CXCL9, CXCL10, CXCL11, CXCL12, FGF-19, and FGF-23 in a biological sample obtained from the subject prior to a combination therapy (pre-treatment),
   (b) measuring levels of the one or more proteins in a biological sample obtained from the subject after initiation of the combination therapy (post-treatment),
   (c) calculating a ratio of the post-treatment level to the pre-treatment level for each of the one or more proteins measured,
   wherein increased ratios, as compared to a control, of the one or more proteins measured are predictive that the subject is likely to respond to the combination therapy; and
      wherein the antagonist is not atezolizumab or CS-1001.
2. The method of Hi, wherein the cancer is selected from the group consisting of: an endometrial cancer, a non-small cell lung cancer (NSCLC), a renal cell carcinoma (RCC), a urothelial cancer, a head and neck cancer, a melanoma, a hepatocellular carcinoma, a breast cancer, an ovarian cancer, a gastric cancer, a colorectal cancer, a bladder cancer, a glioblastoma, a biliary tract cancer, a glioma, Merkel cell carcinoma, Hodgkin lymphoma, non-Hodgkin lymphoma, a cervical cancer, an advanced or refractory solid tumor, a small cell lung cancer, a non-squamous non-small cell lung cancer, desmoplastic melanoma, a pediatric advanced solid tumor or lymphoma, a mesothelin-positive pleural mesothelioma, an esophageal cancer, an anal cancer, a salivary cancer, a prostate cancer, a carcinoid tumor, a primitive neuroectodermal tumor (pNET), and a thyroid cancer.
3. The method of [1] or [2], wherein the biological sample obtained pre-treatment and the biological sample obtained post-treatment from the subject are a blood sample, a serum sample, or a plasma sample.
4. The method of [3], wherein the biological sample is a serum sample.
5. The method of any one of [1] to [4], wherein the one or more proteins are selected from the group consisting of: IFN-γ, CXCL9, CXCL10, and CXCL11.
6. The method of any one of [1] to [4], wherein the one or more proteins are CXCL9 and/or CXCL10.
7. The method of any one of [1] to [4], wherein the one or more proteins are FGF-19 and/or FGF-23.
8. The method of any one of [1] to [7], wherein the pharmaceutically acceptable salt of lenvatinib is lenvatinib mesylate.
9. The method of any one of [1] to [8], wherein the PD-1 antagonist is an antagonist of PD-1.
10. The method of [9], wherein the antagonist of PD-1 is pembrolizumab.
11. The method of [10], wherein lenvatinib or the pharmaceutically acceptable salt thereof is administered daily and pembrolizumab is administered every 3 weeks or every 6 weeks.
12. The method of any one of [1] to [11], wherein the post-treatment biological sample is obtained from the subject at least 1 week after combination therapy initiation.
13. The method of [12], wherein the post-treatment biological sample is obtained from the subject 1 week to 24 months after combination therapy initiation.
14. The method of [13], wherein the post-treatment biological sample is obtained from the subject 1 week to 4 weeks after combination therapy initiation.
15. The method of any one of [1] to [14], wherein the cancer is an endometrial cancer.
16. The method of [15], wherein the endometrial cancer is an advanced endometrial cancer.
17. The method of any one of [1] to [16], further comprising continuing the combination therapy to the subject who is predicted to respond or predicted to be likely to respond to the combination therapy.
18. A method of treating a human subject having a cancer comprising the step of:
   administering a combination therapy comprising lenvatinib or a pharmaceutically acceptable salt thereof and a PD-1 antagonist to the human subject determined to have an increased ratio of one or more proteins selected from the group consisting of IFN-γ, IL-10, CXCL9, CXCL10, CXCL11, CXCL12, FGF-19, and FGF-23,
   wherein the ratio of the one or more proteins is obtained by having measured the level of the one or more proteins in a biological sample obtained from the human subject prior to administering the combination therapy (pre-treatment) and the level of the one or more proteins in a biological sample is obtained after administration of the combination therapy and having determined the ratio; and
   wherein the PD-1 antagonist is not atezolizumab or CS-1001.
19. The method of [18], wherein the cancer is selected from the group consisting of: an endometrial cancer, a non-small cell lung cancer (NSCLC), a renal cell carcinoma (RCC), a urothelial cancer, a head and neck cancer, a melanoma, a bladder cancer, a hepatocellular carcinoma, a breast cancer, an ovarian cancer, a gastric cancer, a colorectal cancer, a glioblastoma, a biliary tract cancer, a glioma, Merkel cell carcinoma, Hodgkin lymphoma, non-Hodgkin lymphoma, a cervical cancer, an advanced or refractory solid tumor, a small cell lung cancer, a non-squamous non-small cell lung cancer, desmoplastic melanoma, a pediatric advanced solid tumor or lymphoma, a mesothelin-positive pleural mesothelioma, an esophageal cancer, an anal cancer, a salivary cancer, a prostate cancer, a carcinoid tumor, a primitive neuroectodermal tumor (pNET), and a thyroid cancer.
20. The method of [18] or [19], wherein the biological sample obtained pre-treatment and the biological sample obtained post-treatment are a blood sample, a serum sample, or a plasma sample.
21. The method of [20], wherein the biological sample is a serum sample or a blood sample.
22. The method of any one of [18] to [21], wherein the one or more proteins are selected from the group consisting of: IFN-γ, CXCL9, CXCL10, and CXCL11.
23. The method of any one of [18] to [21], wherein the one or more proteins are CXCL9 and/or CXCL10.
24. The method of any one of [18] to [21], wherein the one or more proteins are FGF-19 and/or FGF-23.
25. The method of any one of [18] to [24], wherein the pharmaceutically acceptable salt of lenvatinib is lenvatinib mesylate.
26. The method of any one of [18] to [25], wherein the PD-1 antagonist is an antagonist of PD-1.
27. The method of [26], wherein the antagonist of PD-1 is pembrolizumab.
28. The method of [27], wherein lenvatinib or a pharmaceutically acceptable salt thereof is administered to the subject daily and pembrolizumab is administered to the subject every 3 weeks or every 6 weeks.
29. The method of any one of [18] to [28], wherein the post-treatment biological sample is obtained from the subject at least 1 week after combination therapy initiation.
30. The method of [29], wherein the post-treatment biological sample is obtained from the subject 1 week to 24 months after combination therapy initiation.
31. The method of [30], wherein the post-treatment biological sample is obtained from the subject 1 week to 4 weeks after combination therapy initiation.
32. The method of any one of [18] to [31], wherein the cancer is an endometrial cancer.
33. The method of [32], wherein the endometrial cancer is an advanced endometrial cancer.
34. The method of any one of [1] to [33], wherein the one or more proteins is IFN-y.
35. The method of any one of [1] to [33], wherein the one or more proteins is IL-10.
36. The method of any one of [1] to [33], wherein the one or more proteins is CXCL9.
37. The method of any one of [1] to [33], wherein the one or more proteins is CXCL10.
38. The method of any one of [1] to [33], wherein the one or more proteins is CXCL11.
39. The method of any one of [1] to [33], wherein the one or more proteins is CXCL12.
40. The method of any one of [1] to [33], wherein the one or more proteins is FGF-19.
41. The method of any one of [1] to [33], wherein the one or more proteins is FGF-23.
42. The method of any one of [1] to [33], wherein the one or more proteins are one protein selected from the group consisting of: IFN-γ, IL-10, CXCL9, CXCL10, CXCL11, CXCL12, FGF-19 and FGF-23.
43. The method of any one of [1] to [33], wherein the one or more proteins are at least three proteins selected from the group consisting of: IFN-γ, IL-10, CXCL9, CXCL10, CXCL11, CXCL12, FGF-19 and FGF-23.
45. The method of any one of [1] to [33], wherein the one or more proteins are at least three proteins selected from the group consisting of: IFN-γ, IL-10, CXCL9, CXCL10, CXCL11, CXCL12, FGF-19 and FGF-23.
46. The method of any one of [1] to [33], wherein the one or more proteins are at least three proteins selected from the group consisting of: IFN-γ, IL-10, CXCL9,

CXCL10, CXCL11, CXCL12, FGF-19 and FGF-23.
47. The method of any one of [1] to [33], wherein the one or more proteins are at least three proteins selected from the group consisting of: IFN-γ, IL-10, CXCL9, CXCL10, CXCL11, CXCL12, FGF-19 and FGF-23.
48. The method of any one of [1] to [33], wherein the one or more proteins are at least three proteins selected from the group consisting of: IFN-γ, IL-10, CXCL9, CXCL10, CXCL11, CXCL12, FGF-19 and FGF-23.
49. The method of any one of [1] to [33], wherein the one or more proteins are at least three proteins selected from the group consisting of: IFN-γ, IL-10, CXCL9, CXCL10, CXCL11, CXCL12, FGF-19 and FGF-23.

### REFERENCES

All references cited herein are incorporated by reference to the same extent as if each individual publication, database entry (e.g. GenBank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. GenBank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.
1. Sharpe, A. H, Wherry, E. J., Ahmed R., and Freeman G J “The function of programmed cell death 1 and its ligands in regulating autoimmunity and infection,” Nature Immunology (2007); 8: 239-245.
2. Dong H et al., “Tumor-associated B7-H1 promotes T-cell apoptosis: a potential mechanism of immune evasion,” Nat. Med. 2002 8(8): 793-800.
3. Yang et al., “PD-1 interaction contributes to the functional suppression of T-cell responses to human uveal melanoma cells in vitro,” Invest. Ophthalmol. Vis. Sci. 2008 49(6) 2518-2525 (2008).
4. Ghebeh et al., “The B7-H1 (PD-L1) T lymphocyte-inhibitory molecule is expressed in breast cancer patients with infiltrating ductal carcinoma: correlation with important high-risk prognostic factors,” Neoplasia (2006) 8: 190-198.
5. Hamanishi J et al., “Programmed cell death 1 ligand 1 and tumor-infiltrating CD8+T lymphocytes are prognostic factors of human ovarian cancer,” Proceeding of the National Academy of Sciences USA 104: 3360-3365 (2007).
6. Thompson RH et al., “Significance of B7-H1 overexpression in kidney cancer. Clinical Genitourin Cancer 5: 206-211 (2006).
7. Nomi, T. Sho, M., Akahori, T., et al., “Clinical significance and therapeutic potential of the programmed death-1 ligand/programmed death-1 pathway in human pancreatic cancer,” Clinical Cancer Research (2007)13: 2151-2157.
8. Ohigashi Yet al., “Clinical significance of programmed death-1 ligand-1 and programmed death-1 ligand 2 expression in human esophageal cancer,” Clin. Cancer Research (2005) 11: 2947-2953.
9. Inman et al., “PD-L1 (B7-H1) expression by urothelial carcinoma of the bladder and BCG-induced granulomata: associations with localized stage progression,” Cancer (2007) 109: 1499-1505.
10. Shimauchi T et al., “Augmented expression of programmed death-1 in both neoplasmatic and nonneoplastic CD4+T-cells in adult T-cell Leukemia/Lymphoma,” Int. J. Cancer (2007) 121: 2585-2590.
11. Gao et al., “Overexpression of PD-L1 significantly associates with tumor aggressiveness and postoperative recurrence in human hepatocellular carcinoma,” Clinical Cancer Research (2009) 15: 971-979.
12. Nakanishi J., “Overexpression of B7-H1 (PD-L1) significantly associates with tumor grade and postoperative prognosis in human urothelial cancers,” Cancer Immunol. Immunother. (2007) 56: 1173-1182.
13. Hino et al., “Tumor cell expression of programmed cell death-1 is a prognostic factor for malignant melanoma,” Cancer (2010) 116(7): 1757-66.

14. Ghebeh H., “Foxp3+Tregs and B7-H1+/PD-1+T lymphocytes co-infiltrate the tumor tissues of high-risk breast cancer patients: implication for immunotherapy,” BMC Cancer. (2008) 8: 57.
15. Ahmadzadeh M et al., “Tumor antigen-specific CD8 T cells infiltrating the tumor express high levels of PD-1 and are functionally impaired,” Blood (2009) 114: 1537-1544.
16. Thompson RH et al., “PD-1 is expressed by tumor infiltrating cells and is associated with poor outcome for patients with renal carcinoma,” Clinical Cancer Research (2007) 15: 1757-1761.
17. US Patent Application Publication No. 2004-053908.
18. US Patent Application Publication No. 2004-253205.
19. US Patent Application Publication No. 2010-105031.
20. US Patent Application Publication No. 2009-209580.
21. US Patent Application Publication No. 2009-264464.
22. US Patent Application Publication No. 2004-259834.
23. Iwai et al., PNAS, 2002, 99 (19): 12293-7.
24. WO 2015/094995
25. WO 2015/094992
26. WO 2015/ 094996
27. WO 2016/094377

1. A method of predicting the response of a human subject having or suspected of having at least one cancer to a combination therapy comprising lenvatinib or a pharmaceutically acceptable salt thereof and a Programmed Cell Death 1 protein (PD-1) antagonist, the method comprising:
(a) measuring levels of one or more proteins selected from the group consisting of: IFN-γ, IL-10, CXCL9, CXCL10, CXCL11, CXCL12, FGF-19, and FGF-23 in a biological sample obtained from the subject prior to a combination therapy (pre-treatment),
(b) measuring levels of the one or more proteins in a biological sample obtained from the subject after initiation of the combination therapy (post-treatment),
(c) calculating a ratio of the post-treatment level to the pre-treatment level for each of the one or more proteins measured,
wherein increased ratios, as compared to a control, of the one or more proteins measured are predictive that the subject is likely to respond to the combination therapy; and
wherein the antagonist is not atezolizumab or CS-1001.

2. The method of claim 1, wherein the cancer is selected from the group consisting of: an endometrial cancer, a non-small cell lung cancer (NSCLC), a renal cell carcinoma (RCC), a urothelial cancer, a head and neck cancer, a melanoma, a hepatocellular carcinoma, a breast cancer, an ovarian cancer, a gastric cancer, a colorectal cancer, a bladder cancer, a glioblastoma, a biliary tract cancer, a glioma, Merkel cell carcinoma, Hodgkin lymphoma, non-Hodgkin lymphoma, a cervical cancer, an advanced or refractory solid tumor, a small cell lung cancer, a non-squamous non-small cell lung cancer, desmoplastic melanoma, a pediatric advanced solid tumor or lymphoma, a mesothelin-positive pleural mesothelioma, an esophageal cancer, an anal cancer, a salivary cancer, a prostate cancer, a carcinoid tumor, a primitive neuroectodermal tumor (pNET), and a thyroid cancer.

3. The method of claim 1, wherein the biological sample obtained pre-treatment and the biological sample obtained post-treatment from the subject are a blood sample, a serum sample, or a plasma sample.

4. The method of claim 3, wherein the biological sample is a serum sample.

5. The method of claim 1, wherein the one or more proteins are selected from the group consisting of: IFN-γ, CXCL9, CXCL10, and CXCL11.

6. The method of claim 1, wherein the one or more proteins are CXCL9 and/or CXCL10.

7. The method of claim 1, wherein the one or more proteins are FGF-19 and/or FGF-23.

8. The method of claim 1, wherein the pharmaceutically acceptable salt of lenvatinib is lenvatinib mesylate.

9. The method of claim 1, wherein the PD-1 antagonist is an antagonist of PD-1.

10. The method of claim 9, wherein the antagonist of PD-1 is pembrolizumab.

11. The method of claim 10, wherein lenvatinib or the pharmaceutically acceptable salt thereof is administered daily and pembrolizumab is administered every 3 weeks or every 6 weeks.

12. The method of claim 1, wherein the post-treatment biological sample is obtained from the subject at least 1 week after combination therapy initiation.

13. The method of claim 12, wherein the post-treatment biological sample is obtained from the subject 1 week to 24 months after combination therapy initiation.

14. The method of claim 13, wherein the post-treatment biological sample is obtained from the subject 1 week to 4 weeks after combination therapy initiation.

15. The method of claim 1, wherein the cancer is an endometrial cancer.

16. The method of claim 15, wherein the endometrial cancer is an advanced endometrial cancer.

17. The method of claim 1, further comprising continuing the combination therapy to the subject who is predicted to respond or predicted to be likely to respond to the combination therapy.

18. A method of treating a human subject having a cancer comprising the step of:
(a) administering a combination therapy comprising lenvatinib or a pharmaceutically acceptable salt thereof and a PD-1 antagonist to the human subject determined to have an increased ratio of one or more proteins selected from the group consisting of IFN-γ, IL-10, CXCL9, CXCL10, CXCL11, CXCL12, FGF-19, and FGF-23,
wherein the ratio of the one or more proteins is obtained by having measured the level of the one or more proteins in a biological sample obtained from the human subject prior to administering the combination therapy (pre-treatment) and the level of the one or more proteins in a biological sample is obtained after administration of the combination therapy and having determined the ratio; and
wherein the PD-1 antagonist is not atezolizumab or CS-1001.

19. The method of claim 18, wherein the cancer is selected from the group consisting of: an endometrial cancer, a non-small cell lung cancer (NSCLC), a renal cell carcinoma (RCC), a urothelial cancer, a head and neck cancer, a melanoma, a bladder cancer, a hepatocellular carcinoma, a breast cancer, an ovarian cancer, a gastric cancer, a colorectal cancer, a glioblastoma, a biliary tract cancer, a glioma, Merkel cell carcinoma, Hodgkin lymphoma, non-Hodgkin lymphoma, a cervical cancer, an advanced or refractory solid tumor, a small cell lung cancer, a non-squamous non-small cell lung cancer, desmoplastic melanoma, a pediatric advanced solid tumor or lymphoma, a mesothelin-positive pleural mesothelioma, an esophageal cancer, an anal cancer, a salivary cancer, a prostate cancer, a carcinoid tumor, a primitive neuroectodermal tumor (pNET), and a thyroid cancer.

20. The method of claim 18, wherein the biological sample obtained pre-treatment and the biological sample obtained post-treatment are a blood sample, a serum sample, or a plasma sample.

21. The method of claim 20, wherein the biological sample is a serum sample or a blood sample.

22. The method of claim 18, wherein the one or more proteins are selected from the group consisting of: IFN-γ, CXCL9, CXCL10, and CXCL11.

23. The method of claim 18, wherein the one or more proteins are CXCL9 and/or CXCL10.

24. The method of claim 18, wherein the one or more proteins are FGF-19 and/or FGF-23.

25. The method of claim 18, wherein the pharmaceutically acceptable salt of lenvatinib is lenvatinib mesylate.

26. The method of claim 18, wherein the PD-1 antagonist is an antagonist of PD-1.

27. The method of claim 26, wherein the antagonist of PD-1 is pembrolizumab.

28. The method of claim 27, wherein lenvatinib or a pharmaceutically acceptable salt thereof is administered to the subject daily and pembrolizumab is administered to the subject every 3 weeks or every 6 weeks.

29. The method of claim 18, wherein the post-treatment biological sample is obtained from the subject at least 1 week after combination therapy initiation.

30. The method of claim 29, wherein the post-treatment biological sample is obtained from the subject 1 week to 24 months after combination therapy initiation.

31. The method of claim 30, wherein the post-treatment biological sample is obtained from the subject 1 week to 4 weeks after combination therapy initiation.

32. The method of claim 18, wherein the cancer is an endometrial cancer.

33. The method of claim 32, wherein the endometrial cancer is an advanced endometrial cancer.
